(19) United States
(12) Patent Application Publication
(10) Pub. No.: US 2024/0374727 A1
(43) Pub. Date: Nov. 14, 2024

(54) BINDING DOMAIN

(71) Applicant: UCL BUSINESS LTD, London (GB)

(72) Inventors: Marc Mansour, London (GB); Martin Pulé, London (GB); Paul Maciocia, London (GB); Patrycja Wawrzyniecka, London (GB)

(21) Appl. No.: 18/690,416

(22) PCT Filed: Sep. 9, 2022
(86) PCT No.: PCT/GB2022/052297

§ 371 Date: Mar. 8, 2024

(30) Foreign Application Priority Data
Sep. 10, 2021 (GB) 2112923.4

(51) Int. Cl. A61K 39/00; A61P 35/00; C07K 14/705; C07K 14/725; C07K 16/28; C12N 5/0783

(52) U.S. Cl. CPC A61K 39/464421; A61K 39/4611; A61K 39/4613; A61K 39/4631; A61P 35/00; C07K 14/70507; C07K 14/7051; C07K 14/70517; C07K 14/70521; C07K 14/70578; C07K 14/70596; C07K 16/2866; C12N 5/0636; C07K 2319/30; C12N 2510/00

(57) ABSTRACT

The present invention relates to CCR9 binding domains. In particular, the invention provides chimeric antigen receptors (CARs) comprising such CCR9 binding domains.

### FIELD OF THE INVENTION

The present invention relates to CCR9 binding domains. The present invention also relates to chimeric antigen receptors (CARs) comprising such CCR9 binding domains. CARs targeting CCR9 may be useful in the treatment of cancerous diseases such as T cell subgroup of acute lymphoblastic leukaemia (T-ALL), for example.

### BACKGROUND TO THE INVENTION

The T cell subgroup of acute lymphoblastic leukaemia (T-ALL) accounts for over 30% of the paediatric ALL cases that fail to achieve remission with induction chemotherapy. Such patients require treatment intensification resulting in both short and long-term treatment toxicity.

Furthermore, such patients are more likely to experience disease relapse, where the prognosis remains particularly poor, with 5 year survival of less than 40% in children and <10% in adults, primarily due to a lack of efficacious therapies and an inability to tolerate further rounds of intensive chemotherapy or allogeneic transplant.

While immunotherapeutic approaches in the form of CAR T cells have had a major impact on the outcome of relapsed/refractory B-ALL, T-ALL poses a greater challenge for immunotherapy. The vast majority of cell surface antigens are shared between T-ALL cells and normal T cells, which poses two major problems.

Firstly, expression of a CAR directed to an antigen already expressed on the engineered T cells themselves will lead to fratricide and T cell exhaustion, impairing CAR T cell production. Secondly, unlike the B cell aplasia that occurs in B-ALL patients treated with CD19-directed CAR T cell therapy, T cell ablation is prohibitively immunosuppressive to patients.

### SUMMARY OF THE INVENTION

The present inventors have demonstrated that C-C Chemokine receptor 9 (CCR9) is overexpressed in acute and chronic T cell lineage leukaemia, and CCR9 is found to be highly expressed in relapsed/refractory T-ALL cases.

The present inventors have further generated novel CCR9 antibodies, scFVs and CARs.

Thus, in one aspect, the present invention provides a chimeric antigen receptor (CAR) comprising a CCR9 binding domain.

Suitably, the CCR9 binding domain may comprise CDRs with the following sequences:

(SEQ ID NO: 1)
CDR1-GFSVASYD
(SEQ ID NO: 2)
CDR2-IWANGNT
(SEQ ID NO: 3)
CDR3-TRGGFAY
(SEQ ID NO: 4)
CDR4-QSLVHNNGNTY
(SEQ ID NO: 5)
CDR5-KVS
(SEQ ID NO: 6)
CDR6-GQGTQYPT.

One or more of the CDRs may comprise one, two or three amino acid mutations.

Suitably, the CCR9 binding domain may comprise a variable heavy chain (VH) region having the sequence shown as SEQ ID NO: 7 or a variant having at least 80% sequence identity thereto and a variable light chain (VL) region having the sequence shown as SEQ ID NO: 8 or a variant of having at least 80% sequence identity thereto.

Suitably, the CCR9 binding domain may comprise the sequence shown as SEQ ID NO: 10 or a variant thereof having at least 80% sequence identity.

The CAR may comprise a transmembrane domain selected from the group comprising: a CD8a transmembrane domain; a CD28 transmembrane domain; or a Tyrp-1 transmembrane domain.

The CAR may comprise a transmembrane domain comprising one of the sequences selected from the group comprising: SEQ ID NO: 12; SEQ ID NO: 13; or SEQ ID NO: 14; or a variant thereof having at least 80% sequence identity.

Suitably, the CCR9 binding domain and the transmembrane domain may be connected by a spacer.

The spacer may comprise one of the following: an IgG1 Fc domain; an IgG1 hinge; an IgG1 hinge-CD8 stalk; a CD8 stalk; a CD2 ectodomain; a CD34 ectodomain; or COMP.

The spacer may comprise one of the sequences selected from the group comprising: SEQ ID NO: 15; SEQ ID NO: 16; SEQ ID NO: 17; SEQ ID NO: 18; SEQ ID NO: 19; SEQ ID NO: 20; SEQ ID NO: 21; or SEQ ID NO: 22; or a variant thereof having at least 80% sequence identity. The CAR may comprise an intracellular T cell signalling domain.

The intracellular T cell signalling domain may comprise one or more of the following endodomains: CD28 endodomain; 41BB endodomain; OX40 endodomain and CD3-Zeta endodomain.

The intracellular T cell signalling domain may comprise one or more of the sequences selected from the group comprising: SEQ ID NO: 25; SEQ ID NO: 26; SEQ ID NO: 27; SEQ ID NO: 28; SEQ ID NO: 29; SEQ ID NO: 30; or SEQ ID NO: 31; or a variant thereof having at least 80% sequence identity.

Suitably, the CAR may comprise the sequence shown as SEQ ID NO: 35 or a variant thereof which has at least 80% sequence identity thereto but retains the capacity to i) bind CCR9 and ii) induce T cell signalling.

In another aspect, the present invention provides a polynucleotide comprising a nucleic acid sequence encoding a CAR according to the invention.

In a further aspect, the present invention provides a vector which comprises a polynucleotide according to the invention.

In a further aspect, the present invention provides a cell comprising a CAR according to the invention.

In another aspect, the present invention provides a cell comprising a polynucleotide or a vector according to the invention.

The cell may be an immune cell, such as a T cell or an NK cell.

In a further aspect, the present invention provides a method for making a cell according to the invention, which comprises the step of introducing a polynucleotide or a vector according to the invention into said cell.

In a further aspect, the present invention provides a pharmaceutical composition which comprises a CAR, a polynucleotide, a vector, or a cell according the invention, together with a pharmaceutically acceptable carrier, diluent or excipient.

In a further aspect, the present invention provides a CAR, a polynucleotide, a vector, or a cell according the invention, or a pharmaceutical composition according to the invention, for use as a medicament in the treatment of a disease.

In another aspect, the present invention provides a method for treating a disease, which comprises the step of administering a CAR, a polynucleotide, a vector, a cell or a pharmaceutical composition according to the invention to a subject.

In a further aspect, the present invention provides a use of a CAR, a polynucleotide, a vector, a cell, or a pharmaceutical composition according to the invention in the manufacture of a medicament for treating a disease.

The disease may be cancer.

The cancer may be T cell subgroup of acute lymphoblastic leukaemia (T-ALL), prostate, breast, ovarian, non-small cell lung, hepatocellular carcinoma, pancreatic, melanoma, colon, nasopharyngeal carcinoma, Enteropathy-associated T cell lymphoma, hepatosplenic T cell lymphoma, diffuse large B cell lymphoma or mediastinal large B cell lymphoma.

The cancer may be T cell subgroup of acute lymphoblastic leukaemia (T-ALL). Suitably, the T-ALL may be relapsed and/or refractory (r/r) T-ALL.

The disease may be inflammatory bowel disease, Crohn's disease, coeliac disease or graft versus host disease.

### BRIEF DESCRIPTION OF THE FIGURES

FIG. 1: Flow cytometry of CCR9 cell surface expression from 3 T-ALL cell lines, 8 relapsed and 12 primary refractory paediatric T-ALL cases. Dotted line signifies negative control MFI. Representative flow cytometry plots shown in right panels. Mouse IgG2a PE anti-human CCR9 antibody from Biolegend, clone L053E8.

FIG. 2: Only a minor fraction of peripheral blood T cells are positive for CCR9. Flow cytometry of peripheral blood mononuclear cells from a normal donor showing CCR9+ cells accounting for approx. 4% of the CD7+ T cell population. Mouse IgG2a PE anti-human CCR9 antibody from Biolegend, clone L053E8.

FIG. 3: Clone 9G7 specifically binds as a scFv. Flow cytometry of anti-CCR9 scFv (Y-axis) against wild-type P12-Ichikawa T-ALL cells (blue) and P12-Ichikawa CCR9 knockout (KO) cells (red).

FIG. 4: 9G7 CCR9 directed CAR T cells specifically kill CCR9+ T-ALL cell lines (P12-Ichikawa and PF382), but not their isogenic CCR9 knockout counterparts. CD19 is used as control.

FIG. 5: Cytokine comparison of CCR9 CAR T cells at 48 hours measured from 1:8 E: T ratio by ELISA.

FIG. 6: 9G7 CCR9 CAR T cells prolong survival of NSG mice engrafted with MOLT4 T-ALL cells (top panel) n=4 mice in each group. 9G7 CCR9 CAR T cells prevent dissemination of a primary T-ALL PDX into the peripheral blood (bottom panel).

FIG. 7: Anti-CCR9 CAR has potent anti-leukemic activity in patient-derived xenograft models of T-ALL (a) Flow diagram of PDX model 1, n=4/group (b) Flow cytometry of CCR9 expression in blasts of PDX model 1 (c) Serial bleeds of mice, % tumor of total CD45+ cells (d) Survival of mice in PDX model 1 (e) Spleen mass at necropsy in PDX model 1 (f) Tumor in spleen, % of total CD45+ cells (g) Tumor in marrow, % of total CD45+ cells (h) Flow diagram of PDX model 2, n=5 (NT), 3 (CAR19) and 5 (CARCCR9) (i) Flow cytometry of CCR9 expression in blasts of PDX model 2 (j) Serial bleeds of mice in PDX model 2, % tumor of total CD45+ cells (k) Survival of mice in PDX model 2 (I) Spleen mass at necropsy in PDX model 2 (m) Tumor in spleen in PDX model 2, % of total CD45+ cells (n) T cells in spleen in PDX model 2, % of total CD45+ cells (o) T cells and tumor in marrow of CARCCR9 recipients in PDX model 2, % of total CD45+ cells

FIG. 8: Anti-CCR9 CAR has potent anti-leukemic activity in patient-derived xenograft models of T-ALL with low CCR9 antigen density (a) Flow diagram of low density PDX models (b-e) PDX models 3-7. CCR9 antigen density in PDX blasts before injection, molecules per cell (left), leukemic burden in peripheral blood in PDX models (centre) and survival curves of animals in PDX models (right). n=5/group in all models.

### DETAILED DESCRIPTION OF THE INVENTION

### CCR9 Binding Domain

CCR9 (also referred to as cluster of differentiation 199/CDw199) is a member of the beta chemokine receptor family. It is a seven transmembrane protein and its ligand is CCL25.CCR9 is overexpressed in acute and chronic T cell lineage leukaemia, and is found to be highly expressed in relapsed/refractory T-ALL cases.

An illustrative CCR9 amino acid sequence is the human CCR9 sequence designated by UniProt accession number P51686-1. An illustrative amino acid sequence for CCR9 is shown as SEQ ID NO: 11. Suitably, the CCR9 amino acid sequence may comprise the sequence shown as SEQ ID NO: 11.

CCR9
(SEQ ID NO: 11)
MTPTDFTSPIPNMADDYGSESTSSMEDYVNFNFTDFYCEKNNVRQFASHF
LPPLYWLVFIVGALGNSLVILVYWYCTRVKTMTDMFLLNLAIADLLFLVT
LPFWAIAAADQWKFQTFMCKVVNSMYKMNFYSCVLLIMCISVDRYIAIAQ
AMRAHTWREKRLLYSKMVCFTIWVLAAALCIPEILYSQIKEESGIAICTM
VYPSDESTKLKSAVLTLKVILGFFLPFVVMACCYTIIIHTLIQAKKSSKH
KALKVTITVLTVFVLSQFPYNCILLVQTIDAYAMFISNCAVSTNIDICFQ
VTQTIAFFHSCLNPVLYVFVGERFRRDLVKTLKNLGCISQAQWVSFTRRE
GSLKLSSMLLETTSGALSL

Suitably, the CCR9 amino acid sequence may comprise the sequence shown as SEQ ID NO: 11 or a variant having at least 80%, 85%, 90%, 95%, 98% or 99% sequence identity thereto.

An alternative isoform of human CCR9 (isoform 2, P51686-2), lacks amino acids 1-12 of SEQ ID NO: 11.

An illustrative murine CCR9 amino acid sequence is provided by UniProt accession number Q9WUT7.

The antigen-binding domains described herein are able to specifically bind to CCR9.

Thus, the present invention provides a CCR9 binding domain that is capable of binding to CCR9.

Suitably, the CCR9 binding domain may comprise a heavy chain variable (VH) region comprising complementarity determining region (CDR) 1, CDR2 and CDR3 sequences.

Suitably, the CCR9 binding domain may comprise a light chain variable (VL) region comprising CDR4, CDR5 and CDR6 sequences.

CDR4, CDR5 and CDR6 may be presented or re-numbered as CDR1, CDR2 and CDR3 of the VL region, respectively. Therefore, the CDRs of the VH region may be numbered as VH CDR1, CDR2 and CDR3, and the CDRs of the VL region may be numbered as VL CDR1, CDR2 and CDR3.

Thus, a CCR9 binding domain may be presented as comprising: CDR1, CDR2, CDR3, CDR4, CDR5, and CDR6.

Alternatively, a CCR9 binding domain may be presented as comprising:
a VH region having CDRs with the following numbering:
   CDR1
   CDR2
   CDR3; and
a VL region having CDRs with the following numbering:
   CDR1
   CDR2
   CDR3.

Suitably, a VH domain described herein may comprise CDR1, CDR2 and CDR3 sequences described herein. Suitably, a VL domain described herein may comprise CDR1, CDR2 and CDR3 sequences described herein.

In one embodiment, the CCR9 binding domain comprises a VH CDR3 and a VL CDR3 with the following sequences:

(SEQ ID NO: 3)
VH CDR3-TRGGFAY
(SEQ ID NO: 6)
VL CDR3-GQGTQYPT.

One or both of the CDRs may comprise one, two or three amino acid mutations. The CCR9 binding domain according to the invention which comprises CDR(s) comprising one, two or three amino acid mutations compared to SEQ ID NO: 3 and/or SEQ ID NO: 6 maintains the capacity to bind CCR9.

In one embodiment, the CCR9 binding domain comprises CDRs comprising the following sequences:

CDR1-
(SEQ ID NO: 1)
GFSVASYD
CDR2-
(SEQ ID NO: 2)
IWANGNT
CDR3-
(SEQ ID NO: 3)
TRGGFAY
CDR4-
(SEQ ID NO: 4)
QSLVHNNGNTY
CDR5-
(SEQ ID NO: 5)
KVS
CDR6-
(SEQ ID NO: 6)
GQGTQYPT.

In one embodiment, the CCR9 binding domain comprises CDRs consisting of the following sequences:

CDR1-
(SEQ ID NO: 1)
GFSVASYD
CDR2-
(SEQ ID NO: 2)
IWANGNT
CDR3-
(SEQ ID NO: 3)
TRGGFAY
CDR4-
(SEQ ID NO: 4)
QSLVHNNGNTY
CDR5-
(SEQ ID NO: 5)
KVS
CDR6-
(SEQ ID NO: 6)
GQGTQYPT.

In one embodiment, the CCR9 binding domain may comprise a VH region comprising SEQ ID NOs 1-3; and a VL region comprising SEQ ID NOs 4-6.

One or more of the CDRs may comprise one, two or three amino acid mutations. The CCR9 binding domain according to the invention which comprises CDRs as described herein maintains the capacity to bind CCR9.

The CCR9 binding domain may comprise a VH region having the sequence shown as SEQ ID NO: 7 or a variant having at least 80%, 85%, 90%, 95%, 98% or 99% sequence identity thereto (CDR sequences are shown in bold).

The CCR9 binding domain may comprise a VL region having the sequence shown as SEQ ID NO: 8 or a variant of having at least 80%, 85%, 90%, 95%, 98% or 99% sequence identity thereto (CDR sequences are shown in bold).

The CCR9 binding domain may comprise a VH region having the sequence shown as SEQ ID NO: 7 or a variant having at least 80%, 85%, 90%, 95%, 98% or 99% sequence identity thereto; and a VL region having the sequence shown as SEQ ID NO: 8 or a variant of having at least 80%, 85%, 90%, 95%, 98% or 99% sequence identity thereto (CDR sequences are shown in bold).

Suitably, the CCR9 binding domain may comprise a VH region having the sequence shown as SEQ ID NO: 7; and a VL region having the sequence shown as SEQ ID NO: 8.

VH (SEQ ID NO: 7):
QVQLKESGPGLVQPSQTLSLTCTVSGFSVASYDMHWVRLPPGKGLEWMG
IIWANGNTHYNSGLKSRLSISRDTSKSQVFLKMNSLQTEDTAIYFCTRG
GFAYWGQGTLVTVSS
VL (SEQ ID NO: 8):
DVVMTQTPVSLSVSLGGQVSISCRSSQSLVHNNGNTYLSWYLQKPGQSP
QLLIYKVSNRFSGVSDRFSGSGSGTDFTLKISRVEPDDLGVYYCGQGTQ
YPTFGGGTKLELK

Suitably, the VH domain and VL domain may be connected by a peptide linker. Suitable peptide linkers are known in the art. The linker may comprise the sequence:

(SEQ ID NO: 9)
SGGGGSGGGGSGGGGS

Suitably, the CCR9 binding domain comprises the amino acid sequence shown as SEQ ID NO: 10 or a variant thereof having at least 80%, 85%, 90%, 95%, 98% or 99% sequence identity thereto (CDR sequences are shown in bold). A variant CCR9 binding domain according to the present invention maintains the capacity to bind CCR9.

SEQ ID NO: 10:
QVQLKESGPGLVQPSQTLSLTCTVSGFSVASYDMHWVRLPPGKGLEWMG
IIWANGNTHYNSGLKSRLSISRDTSKSQVFLKMNSLQTEDTAIYFCTRG
GFAYWGQGTLVTVSASGGGGSGGGGSGGGGSDVVMTQTPVSLSVSLGGQ
VSISCRSSQSLVHNNGNTYLSWYLQKPGQSPQLLIYKVSNRFSGVSDRF
SGSGSGTDFTLKISRVEPDDLGVYYCGQGTQYPTFGGGTKLELK

In one aspect, the CCR9 binding domain may be capable of binding CCR9.

In one aspect, the CCR9 binding domain may be capable of selectively binding the N-terminal domain of CCR9. Suitably, the CCR9 binding domain may selectively bind to an epitope within the N-terminal domain of CCR9. Suitably, the CCR9 binding domain does not bind to amino acid residues outside of the N-terminal domain of CCR9. Without wishing to be bound by theory, the present inventors consider that CCR9 binding domains that selectively bind the N-terminal domain of CCR9 function more effectively in a CAR than CCR9 binding domains that bind to other domains in CCR9 and/or bind outside the N-terminal domain of CCR9.

In one aspect, the N-terminal domain of CCR9 may consist of amino acid 1-62 of SEQ ID NO: 11. In one aspect, the N-terminal domain of CCR9 may consist of amino acids 1-48 of SEQ ID NO: 11. In one aspect, the N-terminal domain of CCR9 may consist of amino acids 2-22 of SEQ ID NO: 11.

In one aspect, the N-terminal domain of CCR9 may consist of amino acids 1-60, 1-55, 1-50, 1-40, 1-30, or 1-20 of SEQ ID NO: 11.

Suitably, the N-terminal domain of CCR9 may consist of amino acids 1-60, 1-55, or 1-50 of SEQ ID NO: 11.

Suitably, the N-terminal domain of CCR9 may consist of amino acids 1-51 of SEQ ID NO: 11.

Specific binding to the N-terminal domain of CCR9 may be determined using assays which are known in the art. For example, by an ELISA or binding affinity assay using a CCR9 peptide consisting of the N-terminal domain in the absence of the rest of the CCR9 amino acid sequence. Antigen binding domains which specifically bind the N-terminal domain of CCR9 may be generated by immunising a rat or a mouse, for example, using a CCR9 peptide consisting of the N-terminal domain in the absence of the rest of the CCR9 amino acid sequence. Such a strategy will result in the generation of antibodies specific against the N-terminal domain peptide used as the immunogen. Routine methods can then be used to generate scFvs and other antigen-binding molecules, as required.

The term “polypeptide” is used in the conventional sense to mean a series of amino acids, typically L-amino acids, connected one to the other, typically by peptide bonds between the α-amino and carboxyl groups of adjacent amino acids. The term “polypeptide” is used interchangeably with the terms “amino acid sequence”, “peptide” and/or “protein”. The term “residues” is used to refer to amino acids in an amino acid sequence.

The term “variant” refers to a polypeptide that has an equivalent function to the amino acid sequences described herein, but which includes one or more amino acid substitutions, insertions or deletions.

Thus, a CCR9 binding domain may comprise a variant of a CCR9 binding domain sequence as described herein having a least 80%, 85%, 90%, 95%, 98% or 99% sequence identity thereto.

The terms “selectively binds/selectively binding” and “specifically binds/specifically binding” may be used interchangeably herein. Selectively binds/specifically binding may also be used interchangeable with the term “targeting”.

“Heavy chain variable region” or “VH” refers to the fragment of the heavy chain of an antigen-binding domain or antibody that contains three CDRs interposed between flanking stretches known as framework regions, which are more highly conserved than the CDRs and form a scaffold to support the CDRs. “Light chain variable region” or “VL” refers to the fragment of the light chain of an antigen-binding domain or antibody that contains three CDRs interposed between framework regions.

“Complementarity determining region” or “CDR” with regard to antigen-binding domain or antibody or antigen-binding fragment thereof refers to a highly variable loop in the variable region of the heavy chain of the light chain of an antibody. CDRs can interact with the antigen conformation and largely determine binding to the antigen (although some framework regions are known to be involved in binding). The heavy chain variable region and the light chain variable region each contain 3 CDRs (heavy chain CDRs 1, 2 and 3 and light chain CDRs 1, 2 and 3, numbered from the amino to the carboxy terminus).

It may be possible to introduce one or more mutations (substitutions, additions or deletions) into each CDR without negatively affecting CCR9-binding activity. Each CDR may, for example, have one, two or three amino acid mutations. The CCR9 binding domain comprising the one or more of the CDRs which comprise one, two or three amino acid mutations may suitably maintain the capacity to bind CCR9.

The CDRs of the variable regions of a heavy and light chain of an antigen-binding domain or antibody can be predicted from the heavy and light chain variable region sequences of the antibody, using prediction software available in the art, e.g. using the Abysis algorithm, or using the IMGT/V-QUEST software, e.g. the IMGT algorithm (ImMunoGeneTics) which can be found at www.IMGT.org, (see for example Lefranc et al, 2009 NAR 37: D1006-D1012 and Lefranc 2003, Leukemia 17:260-266). CDR regions identified by either algorithm are considered to be equally suitable for use in the invention. CDRs may vary in length, depending on the antigen-binding domain or antibody from which they are predicted and between the heavy and light chains. Thus, the three heavy chain CDRs of an intact antigen-binding domain or antibody may be of different lengths (or may be of the same length) and the three light chain CDRs of an intact antigen-binding domain or antibody may be of different lengths (or may be of the same length). A CDR for example, may range from 2 or 3 amino acids in length to 5, 6, 7, 8, 9, 10, 11, 12, 13, 14, 15, 16, 17, 18, 19 or 20 amino acids in length. Particularly, a CDR may be from 3-14 amino acids in length, e.g. at least 3 amino acids and less than 15 amino acids.

As used herein, “variant” is synonymous with “mutant” and refers to a polynucleotide or amino acid sequence which differs in comparison to the corresponding wild-type sequence. The term “wild-type” is used to mean a gene or protein having a polynucleotide or amino acid sequence respectively, which is identical with the native gene or protein respectively.

Identity comparisons can be conducted by eye, or more usually, with the aid of readily available sequence comparison programs. These commercially available computer programs can calculate % identity between two or more sequences. A suitable computer program for carrying out such an alignment is the GCG Wisconsin Bestfit package (University of Wisconsin, U.S.A.; Devereux et al., 1984, Nucleotide sequences Research 12:387).

Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching. For example, the percentage identity between two polypeptide sequences may be readily determined by BLAST which is freely available at http://blast.ncbi.nlm.nih.gov

Once the software has produced an optimal alignment, it is possible to calculate % identity. The software typically does this as part of the sequence comparison and generates a numerical result.

The sequence may have one or more deletions, insertions or substitutions of amino acid residues which produce a silent change and result in a functionally equivalent molecule. These sequences are encompassed by the present invention. Deliberate amino acid substitutions may be made on the basis of similarity in polarity, charge, solubility, hydrophobicity, hydrophilicity, and/or the amphipathic nature of the residues as long as the activity is retained. For example, negatively charged amino acids include aspartic acid and glutamic acid;

positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Methods for determining binding specificity include, but are not limited to, ELISA, western blot, immunohistochemistry, flow cytometry, Förster resonance energy transfer (FRET), phage display libraries, yeast two-hybrid screens, co-immunoprecipitation, bimolecular fluorescence complementation and tandem affinity purification. Binding affinity can also be determined using methods such as fluorescence quenching, isothermal titration calorimetry.

The capacity of the CCR9 binding domain to bind to CCR9 may be assessed by determining the binding affinity. A quantitative assessment or measurement of binding affinity (e.g. establishing a KD value) may be determined or measured using methods know in the art, such as by surface plasmon resonance, for example by using the Biacore® system. In addition to the equilibrium dissociation constant (KD), the association rate constant (Ka (1/Ms)), and the dissociation rate constant (Kd (1/s)) may also be determined.

Surface Plasmon Resonance (SPR) experiments may be performed with a Biacore T200, for example.

The variants encompassed by the invention have a binding activity that is essential unaltered or improved compared to the corresponding, unaltered polypeptide.

Suitably, the present invention provides a polypeptide comprising a CCR9 binding domain as described herein.

Suitably the polypeptide comprising the CCR9 binding domain may be an antibody or fragment thereof, an antibody conjugate, a chimeric antigen receptor (CAR) or a bispecific T cell engager (BITE).

As used herein, “antibody” means a protein or polypeptide having an antigen binding site or antigen-binding domain which comprises at least one complementarity determining region CDR. The antibody may comprise 3 CDRs and have an antigen binding site which is equivalent to that of a domain antibody (dAb). The antibody may comprise 6 CDRs and have an antigen binding site which is equivalent to that of a classical antibody molecule. The remainder of the polypeptide may be any sequence which provides a suitable scaffold for the antigen binding site and displays it in an appropriate manner for it to bind the antigen. The antibody may be a whole immunoglobulin molecule or a part thereof such as a Fab, F(ab)′₂,

Fv, single chain Fv (ScFv) fragment, Nanobody or single chain variable domain (which may be a VH or VL chain, having 3 CDRs). The antibody may be a bifunctional antibody. The antibody may be non-human, chimeric, humanised or fully human. The antibody may be a monoclonal antibody or a polyclonal antibody. Preferably, the antibody is a monoclonal antibody.

As used herein, an “antibody conjugate” is a molecule composed of an antibody or fragment thereof described herein, linked (i.e. conjugated) to a biologically active cytotoxic payload or drug, such as an anticancer drug.

As used herein, “a bispecific T cell engager” (BiTE) is a T cell activator molecule which is a bi-specific molecule which comprises a CCR9 binding domain as described herein as a first domain, and a T cell activating domain as a second domain. The second domain of the bi-specific T cell engager molecule may bind CD3.

In the context of a CCR9 binding molecule described above, in order to stimulate cell activation the CCR9 binding domain may bind to its cognate antigen (CCR9) with a certain binding profile (for example, with a required binding affinity). Suitably, in order to stimulate T cell activation the CCR9 binding domain may bind to its cognate antigen (CCR9) with a certain binding profile (for example, with a required binding affinity).

### Chimeric Antigen Receptor

The present invention provides a CAR comprising a CCR9 binding domain as defined herein. Chimeric antigen receptors, also known as CARs, chimeric T cell receptors, artificial T cell receptors and chimeric immunoreceptors, are engineered receptors, which graft an arbitrary specificity onto an immune effector cell. In a classical CAR, the specificity of a monoclonal antibody is grafted on to a T cell. CAR-encoding nucleic acids may be transferred to T cells using, for example, retroviral vectors. In this way, a large number of cancer-specific T cells can be generated for adoptive cell transfer. Phase I clinical studies of this approach show efficacy.

A CAR according to the present invention thus comprises a CCR9 binding domain, a transmembrane domain and an intracellular signalling domain.

The target-antigen binding domain of a CAR is commonly fused via a spacer and transmembrane domain to a signalling endodomain, wherein said signalling endodomain is capable of directly transducing an activation signal into the T cell activation signalling cascade. When the CAR binds the target-antigen, this results in the transmission of an activating signal to the T cell it is expressed on. Thus, the CAR of the present invention is able to activate the T cell it is expressed on following binding of the CAR to CCR9 expressed on the surface of target cells.

Suitably, the CCR9 binding domain as defined herein may be fused via a spacer and transmembrane domain to a signalling endodomain.

### Transmembrane Domain

The CAR of the invention may comprise a transmembrane domain which spans the membrane of a cell. The transmembrane domain may be any protein structure which is thermodynamically stable in a membrane. It may comprise a hydrophobic alpha helix. The transmembrane domain may be derived from CD8, CD28, human Tyrp-1 or human IgG.

The transmembrane domain may be derived from CD8, which gives good receptor stability. The transmembrane domain may be derived from any type I transmembrane protein. The transmembrane domain may be a synthetic sequence predicted to form a hydrophobic helix.

As used herein, the term “derived from” refers to the origin or source, and may include naturally occurring, recombinant, unpurified, or purified molecules. The term “derived from” encompasses the terms “originated from,” “obtained from,” “obtainable from,” “isolated from,” and “created from.”

The transmembrane domain may comprise the sequence shown as SEQ ID NO: 12.

(CD8a transmembrane domain)
SEQ ID NO: 12
IYIWAPLAGTCGVLLLSLVIT

The transmembrane domain may comprise the sequence shown as SEQ ID NO: 13.

(CD28 transmembrane domain)
SEQ ID NO: 13
FWVLVVVGGVLACYSLLVTVAFIIFWV

The transmembrane domain may comprise the sequence shown as SEQ ID NO: 14.

(Tyrp-1 transmembrane domain)
SEQ ID NO: 14
IIAIAVVGALLLVALIFGTASYLI

The CAR of the invention may comprise a variant of the sequence shown as SEQ ID NO: 12, 13 or 14 having at least 80% sequence identity, provided that the variant sequence retains the capacity to insert into and span the membrane.

The variant sequence may have at least 80%, 85%, 90%, 95%, 98% or 99% sequence identity to SEQ ID NO: 12, provided that the variant sequence retains the capacity to insert into and span the membrane.

The variant sequence may have at least 80%, 85%, 90%, 95%, 98% or 99% sequence identity to SEQ ID NO: 13, provided that the variant sequence retains the capacity to insert into and span the membrane.

The variant sequence may have at least 80%, 85%, 90%, 95%, 98% or 99% sequence identity to SEQ ID NO: 14, provided that the variant sequence retains the capacity to insert into and span the membrane.

### Spacer

The CAR of the present invention may comprise a spacer sequence to connect the CCR9 binding domain with the transmembrane domain and spatially separate the CCR9 binding domain from the endodomain. A flexible spacer allows the CCR9 binding domain to orient in different directions to enable CCR9 binding.

The spacer sequence may, for example, comprise an lgG1 Fc region, an IgG1 hinge or a CD8 stalk, or a combination thereof. The spacer may alternatively comprise an alternative sequence which has similar length and/or domain spacing properties as an lgG1 Fc region, an lgG1 hinge or a CD8 stalk. The spacer sequence may, for example, comprise a CD2 ectodomain, CD34 ectodomain or COMP.

A human IgG1 spacer may be altered to remove Fc binding motifs.

The spacer of the CAR of the present invention may comprise one or more of the sequences shown as SEQ ID NO: 15, 16, 17, 18, 19, 20, 21 or 22, or a variant thereof having at least 80% sequence identity.

Examples of amino acid sequences for these spacers are given below:

(hinge-CH2CH3 of human IgG1)
SEQ ID NO: 15
AEPKSPDKTHTCPPCPAPPVAGPSVFLFPPKPKDTLMIARTPEVTCVVVDVSHEDPEVKFN
WYVDGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGKKD
SEQ ID NO: 16 (human CD8 stalk):
TTTPAPRPPTPAPTIASQPLSLRPEACRPAAGGAVHTRGLDFACD
SEQ ID NO: 17 (human IgG1 hinge):
AEPKSPDKTHTCPPCPKDPK
(IgG1 Hinge-Fc)
SEQ ID NO: 18
AEPKSPDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPGKKDPK
(IgG1 Hinge-Fc modified to remove Fc receptor recognition motifs)
SEQ ID NO: 19
AEPKSPDKTHTCPPCPAPPVA*GPSVFLFPPKPKDTLMIARTPEVTCVVVDVSHEDPEVKFN
WYVDGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGKKDPK
Modified residues are underlined; * denotes a deletion.
(CD2 ectodomain)
SEQ ID NO: 20
KEITNALETWGALGQDINLDIPSFQMSDDIDDIKWEKTSDKKKIAQFRKEKETFKEKDTYKLF
KNGTLKIKHLKTDDQDIYKVSIYDTKGKNVLEKIFDLKIQERVSKPKISWTCINTTLTCEVMNG
TDPELNLYQDGKHLKLSQRVITHKWTTSLSAKFKCTAGNKVSKESSVEPVSCPEKGLD
(CD34 ectodomain)
SEQ ID NO: 21
SLDNNGTATPELPTQGTFSNVSTNVSYQETTTPSTLGSTSLHPVSQHGNEATTNITETTVKF
TSTSVITSVYGNTNSSVQSQTSVISTVFTTPANVSTPETTLKPSLSPGNVSDLSTTSTSLATS
PTKPYTSSSPILSDIKAEIKCSGIREVKLTQGICLEQNKTSSCAEFKKDRGEGLARVLCGEEQ
ADADAGAQVCSLLLAQSEVRPQCLLLVLANRTEISSKLQLMKKHQSDLKKLGILDFTEQDVA
SHQSYSQKT
(COMP)
SEQ ID NO: 22
DLGPQMLRELQETNAALQDVRELLRQQVREITFLKNTVMECDACG

It is possible to truncate the COMP coiled-coil domain at the N-terminus and retain surface expression. The coiled-coil COMP spacer may therefore comprise or consist of a truncated version of SEQ ID NO: 22, which is truncated at the N-terminus. The truncated COMP may comprise the 5 C-terminal amino acids of SEQ ID NO: 22, i.e. the sequence CDACG (SEQ ID NO: 23). The truncated COMP may comprise 5 to 44 amino acids, for example, at least 5, 10, 15, 20, 25, 30, 35 or 40 amino acids. The truncated COMP may correspond to the C-terminus of SEQ ID NO: 22. For example a truncated COMP comprising 20 amino acids may comprise the sequence QQVREITFLKNTVMECDACG (SEQ ID NO: 24). Truncated COMP may retain the cysteine residue(s) involved in multimerisation. Truncated COMP may retain the capacity to form multimers.

A variant spacer sequence may have at least 80%, 85%, 90%, 95%, 98% or 99% sequence identity to SEQ ID NO: 15.

A variant spacer sequence may have at least 80%, 85%, 90%, 95%, 98% or 99% sequence identity to SEQ ID NO: 16.

A variant spacer sequence may have at least 80%, 85%, 90%, 95%, 98% or 99% sequence identity to SEQ ID NO: 17.

A variant spacer sequence may have at least 80%, 85%, 90%, 95%, 98% or 99% sequence identity to SEQ ID NO: 18.

A variant spacer sequence may have at least 80%, 85%, 90%, 95%, 98% or 99% sequence identity to SEQ ID NO: 19.

A variant spacer sequence may have at least 80%, 85%, 90%, 95%, 98% or 99% sequence identity to SEQ ID NO: 20.

A variant spacer sequence may have at least 80%, 85%, 90%, 95%, 98% or 99% sequence identity to SEQ ID NO: 21.

A variant spacer sequence may have at least 80%, 85%, 90%, 95%, 98% or 99% sequence identity to SEQ ID NO: 22.

### Intracellular Signalling Domain (Endodomain)

The endodomain is the signal-transmission portion of the CAR. After antigen recognition, receptors cluster and a signal is transmitted to the cell. The most commonly used endodomain component is that of CD3-zeta which contains 3 ITAMs. This transmits an activation signal to the T cell after antigen is bound. CD3-zeta may not provide a fully competent activation signal and additional co-stimulatory signalling may be needed. For example, chimeric CD28, 41-BB and OX40 can be used with CD3-Zeta to transmit a proliferative/survival signal, or all three can be used together.

The endodomain of the CAR of the present invention may comprise a CD28 endodomain or an OX40 endodomain or a 41-BB endodomain or a CD3-Zeta endodomain.

The endodomain of the CAR of the present invention may comprise one of more of: a CD28 endodomain and/or an OX40 endodomain and/or a 41-BB endodomain and/or a CD3-Zeta endodomain.

The endodomain of the CAR of the present invention may comprise a CD28 endodomain and an OX40 endodomain and a 41-BB endodomain and a CD3-Zeta endodomain.

The intracellular T cell signalling domain (endodomain) of the CAR of the present invention may comprise one or more of the sequence shown as SEQ ID NO: 25, 26, 27, 28, 29, 30 or 31, or a variant thereof having at least 80% sequence identity.

(CD28 endodomain)
SEQ ID NO: 25
RSKRSRLLHSDYMNMTPRRPGPTRKHYQPYAPPRDFAAY
(OX40 endodomain)
SEQ ID NO: 26
RRDQRLPPDAHKPPGGGSFRTPIQEEQADAHSTLAKI
(41-BB endodomain)
SEQ ID NO: 27
KRGRKKLLYIFKQPFMRPVQTTQEEDGCSCRFPEEEEGGCEL
(CD3 zeta endodomain)
SEQ ID NO: 28
RSRVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR
(CD28Z)
SEQ ID NO: 29
RSKRSRLLHSDYMNMTPRRPGPTRKHYQPYAPPRDFAAYRSRVKFSRSADAPAYQQGQN
QLYNELNLGRREEYDVLDKRRGRDPEMGGKPRRKNPQEGLYNELQKDKMAEAYSEIGMK
GERRRGKGHDGLYQGLSTATKDTYDALHMQALPPR
(CD28OXZ)
SEQ ID NO: 30
RSKRSRLLHSDYMNMTPRRPGPTRKHYQPYAPPRDFAAYRSRDQRLPPDAHKPPGGGSF
RTPIQEEQADAHSTLAKIRVKFSRSADAPAYQQGQNQLYNELNLGRREEYDVLDKRRGRD
PEMGGKPRRKNPQEGLYNELQKDKMAEAYSEIGMKGERRRGKGHDGLYQGLSTATKDTY
DALHMQALPPR
(4-1BB-CD3Z)
SEQ ID NO: 31
KRGRKKLLYIFKQPFMRPVQTTQEEDGCSCRFPEEEEGGCELRVKFSRSADAPAYQQGQN
QLYNELNLGRREEYDVLDKRRGRDPEMGGKPRRKNPQEGLYNELQKDKMAEAYSEIGMK
GERRRGKGHDGLYQGLSTATKDTYDALHMQALPPRA

A variant endodomain sequence may have at least 80%, 85%, 90%, 95%, 98% or 99% sequence identity to SEQ ID NO: 25, provided that the sequence provides an effective intracellular T cell signalling domain.

A variant endodomain sequence may have at least 80%, 85%, 90%, 95%, 98% or 99% sequence identity to SEQ ID NO: 26, provided that the sequence provides an effective intracellular T cell signalling domain.

A variant endodomain sequence may have at least 80%, 85%, 90%, 95%, 98% or 99% sequence identity to SEQ ID NO: 27, provided that the sequence provides an effective intracellular T cell signalling domain.

A variant endodomain sequence may have at least 80%, 85%, 90%, 95%, 98% or 99% sequence identity to SEQ ID NO: 28, provided that the sequence provides an effective intracellular T cell signalling domain.

A variant endodomain sequence may have at least 80%, 85%, 90%, 95%, 98% or 99% sequence identity to SEQ ID NO: 29, provided that the sequence provides an effective intracellular T cell signalling domain.

A variant endodomain sequence may have at least 80%, 85%, 90%, 95%, 98% or 99% sequence identity to SEQ ID NO: 30, provided that the sequence provides an effective intracellular T cell signalling domain.

A variant endodomain sequence may have at least 80%, 85%, 90%, 95%, 98% or 99% sequence identity to SEQ ID NO: 31, provided that the sequence provides an effective intracellular T cell signalling domain.

### Signal Peptide

The CAR of the present invention may comprise a signal peptide so that when the CAR is expressed inside a cell, such as a T cell, the nascent protein is directed to the endoplasmic reticulum and subsequently to the cell surface, where it is expressed.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

The signal peptide may be at the amino terminus of the molecule.

The CAR of the invention may have the general formula:
Signal peptide—CCR9 binding domain—spacer domain—transmembrane domain—intracellular T cell signalling domain(s).

The signal peptide may comprise the SEQ ID NO: 32 or a variant thereof having 5, 4, 3, 2 or 1 amino acid mutations (e.g. insertions, substitutions or additions) provided that the signal peptide still functions to cause cell surface expression of the CAR.

SEQ ID NO: 32:
METDTLLLWVLLLWVPGSTG

The signal peptide of SEQ ID NO: 32 is compact and highly efficient. It is predicted to give about 95% cleavage after the terminal glycine, giving efficient removal by signal peptidase.

### Suicide Genes

Since T cells engraft and are autonomous, a means of selectively deleting CAR T cells in recipients of CAR T cells is desirable. Suicide genes are genetically encodable mechanisms which result in selective destruction of infused T cells in the face of unacceptable toxicity. The earliest clinical experience with suicide genes is with the Herpes Virus Thymidine Kinase (HSV-TK) which renders T cells susceptible to Ganciclovir. HSV-TK is a highly effective suicide gene. However, pre-formed immune responses may restrict its use to clinical settings of considerable immunosuppression such as haploidentical stem cell transplantation. Inducible Caspase 9 (iCasp9) is a suicide gene constructed by replacing the activating domain of Caspase 9 with a modified FKBP12. iCasp9 is activated by an otherwise inert small molecular chemical inducer of dimerization (CID). iCasp9 has been recently tested in the setting of haploidentical HSCT and can abort GvHD. The biggest limitation of iCasp9 is dependence on availability of clinical grade proprietary CID. Both iCasp9 and HSV-TK are intracellular proteins, so when used as the sole transgene, they have been co-expressed with a marker gene to allow selection of transduced cells.

Suitably, a CAR of the present invention may be expressed with a suicide gene.

Suitably, the suicide gene may be iCasp9.

An iCasp9 may comprise the sequence shown as SEQ ID NO: 33 or a variant thereof having at least 80%, 85%, 90%, 95%, 98% or 99% sequence identity.

SEQ ID NO: 33
MLEGVQVETISPGDGRTFPKRGQTCVVHYTGMLEDGKKVDSSRDRNKPF
KFMLGKQEVIRGWEEGVAQMSVGQRAKLTISPDYAYGATGHPGIIPPHA
TLVFDVELLKLESGGGSGVDGFGDVGALESLRGNADLAYILSMEPCGHC
LIINNVNFCRESGLRTRTGSNIDCEKLRRRFSSLHFMVEVKGDLTAKKM
VLALLELAQQDHGALDCCVVVILSHGCQASHLQFPGAVYGTDGCPVSVE
KIVNIFNGTSCPSLGGKPKLFFIQACGGEQKDHGFEVASTSPEDESPGS
NPEPDATPFQEGLRTFDQLDAISSLPTPSDIFVSYSTFPGFVSWRDPKS
GSWYVETLDDIFEQWAHSEDLQSLLLRVANAVSVKGIYKQMPGCFNFLR
KKLFFKTSAS

Another marker/suicide gene is RQR8 which can be detected with the antibody QBEnd10 and expressing cells lysed with the therapeutic antibody Rituximab. Suitably, the CAR of the present invention may be expressed with RQR8 as a suicide gene.

An RQR8 may comprise the sequence shown as SEQ ID NO: 34 or a variant thereof having at least 80%, 85%, 90%, 95%, 98% or 99% sequence identity.

SEQ ID NO: 34
MGTSLLCWMALCLLGADHADACPYSNPSLCSGGGGSELPTQGTFSNVST
NVSPAKPTTTACPYSNPSLCSGGGGSPAPRPPTPAPTIASQPLSLRPEA
CRPAAGGAVHTRGLDFACDIYIWAPLAGTCGVLLLSLVITLYCNHRNRR
RVCKCPRPVV

The suicide gene may be expressed in a single polypeptide with the CAR, for example by using a self-cleaving peptide between the two sequences.

### CCR9 CARs

The CAR of the present invention may comprise a CCR9 binding domain, a CD8 spacer region, a CD8 transmembrane region, and a 41bb intracellular signalling domain and a CD3-zeta chain intracellular signalling domain.

The CAR of the present invention may comprise the sequence shown as SEQ ID NO: 35 or a variant thereof which has at least 80%, 85%, 90%, 95%, 98% or 99% sequence identity thereto but retains the capacity to i) bind CCR9 and ii) induce T cell signalling.

SEQ ID NO: 35
METDTLLLWVLLVWIPGSTGQVQLKESGPGLVQPSQTLSLTCTVSGFSV
ASYDMHWVRLPPGKGLEWMGIIWANGNTHYNSGLKSRLSISRDTSKSQV
FLKMNSLQTEDTAIYFCTRGGFAYWGQGTLVTVSSSGGGGSGGGGSGGG
GSDVVMTQTPVSLSVSLGGQVSISCRSSQSLVHNNGNTYLSWYLQKPGQ
SPQLLIYKVSNRFSGVSDRFSGSGSGTDFTLKISRVEPDDLGVYYCGQG
TQYPTFGGGTKLELKRSDPTTTPAPRPPTPAPTIASQPLSLRPEACRPA
AGGAVHTRGLDFACDIYIWAPLAGTCGVLLLSLVITLYCKRGRKKLLYI
FKQPFMRPVQTTQEEDGCSCRFPEEEEGGCELRVKFSRSADAPAYQQGQ
NQLYNELNLGRREEYDVLDKRRGRDPEMGGKPRRKNPQEGLYNELQKDK
MAEAYSEIGMKGERRRGKGHDGLYQGLSTATKDTYDALHMQALPPR

The CAR of the present invention may comprise the sequence shown as SEQ ID NO: 35 lacking amino acids 1-20 (which correspond to the signal peptide) or a variant thereof which has at least 80%, 85%, 90%, 95%, 98% or 99% sequence identity thereto but retains the capacity to i) bind CCR9 and ii) induce T cell signalling.

### Polynucleotide

In one aspect, the present invention provides a polynucleotide comprising a nucleic acid sequence which encodes a CCR9 binding domain of the present invention.

In one aspect, the present invention provides a polynucleotide comprising a nucleic acid sequence which encodes a CAR of the present invention.

Due to the redundancy of the genetic code, variations in nucleic acid sequences are possible that encode for the same polypeptide. These sequences are encompassed by the present invention. Therefore multiple polynucleotides are envisaged, each with a different nucleic acid sequence but which encodes a polypeptide according to the invention or a further polypeptide as described herein. It is possible to design and produce such nucleic acid sequences without difficulty.

The nucleic acid sequence may be an RNA or DNA sequence or a variant thereof. The term “polynucleotide” includes an RNA or DNA sequence. It may be single or double stranded. It may, for example, be genomic, recombinant, mRNA or cDNA.

The polynucleotide may be codon optimised for production in the host cell of choice.

As used herein, “variant” is synonymous with “mutant” and refers to a polynucleotide or amino acid sequence which differs in comparison to the corresponding wild-type sequence. The term “wild-type” is used to mean a gene or protein having a polynucleotide or amino acid sequence respectively, which is identical with the native gene or protein respectively.

Suitably, the nucleic acid sequence may be operably linked to a heterologous sequence, such as a promoter or regulatory sequence, forming an expression cassette.

The expression cassette may comprise one or more control sequences. Control sequences are sequences that control and regulate transcription and, where appropriate, the translation of the CCR9 binding domain of the present invention or CAR of the present invention, and include promoter sequences, transcriptional regulators encoding sequences, ribosome binding sequences (RBS) and/or transcription terminating sequences. The expression cassette may additionally include an enhancer, which may be adjacent to or distant from the promoter sequence and can function to increase transcription from the same. The expression control sequence may be functional in prokaryotic cells or in eukaryotic cells and organisms, such as mammalian cells. The expression cassette may comprise a promoter. Any promoter may be used in this methodology. In general, it is advantageous to employ a strong promoter functional in eukaryotic cells. The strong promoter may be, but not limited to, the immediate early cytomegalovirus promoter (CMV-IE) of human or murine origin, or optionally having another origin such as the rat or guinea pig.

In more general terms, the promoter has either a viral, or a cellular origin. A strong viral promoter other than CMV-IE that may be usefully employed in the practice of the invention is the early/late promoter of the SV40 virus or the LTR promoter of the Rous sarcoma virus. A strong cellular promoter that may be usefully employed in the practice of the invention is the promoter of a gene of the cytoskeleton, such as e.g. the desmin promoter (Kwissa et al., 2000), or the actin promoter (Miyazaki et al., 1989).

The promoter may be constitutive promoter. The promoter may be a tissue specific promoter. The promoter may be an EF1-alpha, CMV, PGK or MND promoter.

### Vector

The present invention also provides a vector which comprises a polynucleotide according to the present invention. For example, the vector of the invention may comprise a polynucleotide comprising a nucleic acid sequence that encodes a CCR9 binding domain of the invention or a CAR of the invention. Such a vector may be used to introduce the nucleic acid sequence into a host cell so that it expresses and produces a CCR9 binding domain of the invention or a CAR of the invention.

The vector may be any agent capable of delivering or maintaining nucleic acid in a host cell, and includes viral vectors, plasmids, naked nucleic acids, nucleic acids complexed with polypeptide or other molecules and nucleic acids immobilised onto solid phase particles. The vector may, for example, be a plasmid or a viral vector, such as a retroviral vector or a lentiviral vector.

The vector may comprise the nucleic acid sequence encoding the CCR9 binding domain according to the invention or the CAR according to the invention, operably linked to a heterologous sequence, such as a promoter or regulatory sequence. In general, it is advantageous to employ a strong promoter functional in eukaryotic cells. The strong promoter may be, but not limited to, the immediate early cytomegalovirus promoter (CMV-IE) of human or murine origin, or optionally having another origin such as the rat or guinea pig.

In more general terms, the promoter has either a viral, or a cellular origin. A strong viral promoter other than CMV-IE that may be usefully employed in the practice of the invention is the early/late promoter of the SV40 virus or the LTR promoter of the Rous sarcoma virus. A strong cellular promoter that may be usefully employed in the practice of the invention is the promoter of a gene of the cytoskeleton, such as e.g. the desmin promoter (Kwissa et al., 2000), or the actin promoter (Miyazaki et al., 1989).

The promoter may be constitutive promoter. The promoter may be a tissue specific promoter.

The vector may be capable of transfecting or transducing a cell. In one aspect, the vector may be capable of transfecting or transducing a T cell. In one aspect, the vector may be capable of transfecting or transducing an NK cell.

The vector may also comprise a nucleic acid sequence encoding a suicide gene, such as iCasp9 or RQR8.

### Cell

The invention also provides a host cell which comprises a polynucleotide or a vector according to the invention.

The host cell may be capable of producing a CCR9 binding domain of the invention.

The host cell may be capable of producing and/or expressing and/or may comprise a CAR of the invention.

The host cell may be a bacterial, fungal, yeast, plant or animal cell. Suitably the CCR9 binding domain of the invention may be produced in a bacterial, fungal, yeast, plant or animal cell. Suitably, the host cell may be a mammalian cell, such as the human embryonic kidney cell line 293.

The host cell may be any eukaryotic cell capable of expressing a CAR at the cell surface, such as an immunological cell. In particular, the cell may be an immune cell such as a T cell or an NK cell.

T cells or T lymphocytes are a type of lymphocyte that play a central role in cell-mediated immunity. They can be distinguished from other lymphocytes, such as B cells and natural killer cells (NK cells), by the presence of a T cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class Il molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytotoxic T cells (TC cells, or CTLs) destroy virally infected cells and tumour cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+ Treg cells) arise in the thymus and have been linked to interactions between developing T cells with both myeloid (CD11c+) and plasmacytoid (CD123+) dendritic cells that have been activated with TSLP.

Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr1 cells or Th3 cells) may originate during a normal immune response.

Gamma delta T cells (γδ T cells) are T cells that have a TCR that is comprised of one γ (gamma) chain and one δ (delta) chain. Gamma delta T cells are typically less common than αβ T cells. In humans, in 95% of T cells the TCR consists of an alpha (α) chain and a beta (β) chain (encoded by TRA and TRB, respectively). However, in about 5% of T cells the TCR consists of gamma and delta (γ/δ) chains (encoded by TRG and TRD, respectively). Gamma delta T cells are abundant in the gut mucosa. Examples of gamma delta cells include Vγ9Vδ2 T cells.

γδ TCRs are MHC independent and may detect markers of cellular stress expressed by tumours. The γδ TCR may be capable of binding to a phosphoantigen/butyrophilin 3A1 complex; major histocompatibility complex class I chain-related A (MICA); major histocompatibility complex class I chain-related B (MICB); NKG2D ligand 1-6 (ULBP 1-6); CD1c; CD1d; endothelial protein C receptor (EPCR); lipohexapeptides; phycoreythrin or histidyl-tRNA-synthase.

Natural killer T (NKT) cells are a heterogeneous group of T cells that share properties of both T cells and natural killer cells. Many of these cells recognize the non-polymorphic CD1d molecule, an antigen-presenting molecule that binds self and foreign lipids and glycolipids.

Natural killer (NK) cells are a type of cytolytic cell which forms part of the innate immune system. NK cells provide rapid responses to innate signals from virally infected cells in an MHC independent manner.

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The cell of the invention may be any of the cell types mentioned above.

The cell may be a cytolytic immune cell such as a T cell, natural killer (NK) cell or NKT cell. The cell may be a T cell or natural killer (NK) cell. The cell may be a CTL. Suitably, the host cell may be a T cell. Suitably, the host cell may be a NK cell.

Suitably the T cell may produce and/or express and/or comprise the CAR of the invention.

Suitably the NK cell may produce and/or express and/or comprise the CAR of the invention. A T cell capable of expressing a CAR according to the invention may be made by transducing or transfecting a T cell with CAR-encoding nucleic acid. A NK cell capable of expressing a

CAR according to the invention may be made by transducing or transfecting a NK cell with a CAR-encoding nucleic acid.

The cell may either be created ex vivo either from a patient's own peripheral blood (1st party), or in the setting of a haematopoietic stem cell transplant from donor peripheral blood (2nd party), or peripheral blood from an unconnected donor (3rd party). The cell may be from a peripheral blood mononuclear cell (PBMC) sample from the patient or a donor.

Alternatively, cells may be derived from ex vivo differentiation of inducible progenitor cells or embryonic progenitor cells to, for example, T or NK cells. Alternatively, an immortalized T cell line which retains its lytic function and could act as a therapeutic may be used.

The cell may be activated and/or expanded prior to being transduced with the CAR-encoding nucleic acid, for example by treatment with an anti-CD3 monoclonal antibody.

The present invention also provides a method for making a cell according to the invention, which comprises the step of introducing a polynucleotide according to the invention or a vector according to the invention into said cell. The polynucleotide or vector may, for example, be introduced by transduction or transfection in vitro or ex vivo. The cell is then capable of expressing and/or producing a CCR9 binding domain of the invention or a CAR of the invention, when the host cell is cultured under conditions suitable for production of said CCR9 binding molecule or CAR.

Suitably, the CCR9 binding molecule may be harvested from the host cell or supernatant.

CCR9 binding molecule of the invention produced in a cell as set out above may be produced either intracellularly (e.g. in the cytosol, in the periplasma or in inclusion bodies) and then isolated from the host cells and optionally further purified; or they can be produced extracellularly (e.g. in the medium in which the host cells are cultured) and then isolated from the culture medium and optionally further purified.

### Pharmaceutical Composition

The present invention also provides a pharmaceutical composition comprising a polypeptide comprising a CCR9 binding domain of the present invention (for example a CAR of the present invention) or a cell of the invention together with a pharmaceutically acceptable carrier, diluent or excipient, and optionally one or more further pharmaceutically active polypeptides and/or compounds.

The present invention also provides a pharmaceutical composition comprising a polynucleotide of the invention together with a pharmaceutically acceptable carrier, diluent or excipient, and optionally one or more further pharmaceutically active polypeptides and/or compounds.

The present invention also provides a pharmaceutical composition comprising a vector of the invention together with a pharmaceutically acceptable carrier, diluent or excipient, and optionally one or more further pharmaceutically active polypeptides and/or compounds.

The pharmaceutical composition may be administered to a patient.

The pharmaceutical composition may enable delivery and/or maintenance of a polynucleotide of the invention in a host cell or subject. Such delivery suitably enables expression of CCR9 binding domain of the invention or a CAR of the invention in a host cell or subject. The polynucleotide may be DNA, RNA, or mRNA. The pharmaceutical composition may comprise a viral vector, plasmid, naked nucleic acid, nucleic acid complexed with polypeptide or other molecules and nucleic acids immobilised onto solid phase particles. The vector may, for example, be a plasmid or a viral vector, such as a retroviral vector or a lentiviral vector. Other delivery agents include delivery nanoparticles and lipid nanoparticles (LNP).

The form of administration may be for example, be in a form suitable for oral administration, or for parenteral administration (such as by intravenous, intramuscular or subcutaneous injection or intravenous infusion), or intratumorally.

Suitably, such formulations may, for example, be in a form suitable for intravenous infusion.

### Medical Use

The present invention provides a CCR9 binding domain or polypeptide comprises a CCR9 binding domain of the invention for use as a medicament in the treatment of a disease.

The present invention provides a CAR of the invention for use as a medicament in the treatment of a disease.

The present invention provides a cell of the invention for use as a medicament in the treatment of a disease.

The present invention provides a polynucleotide of the invention for use as a medicament in the treatment of a disease.

The present invention provides a vector of the invention for use as a medicament in the treatment of a disease.

The present invention provides a pharmaceutical composition of the invention for use as a medicament in the treatment of a disease.

Suitably, the disease may be associated with CCR9 expression.

Suitably the disease may be cancer or a cancerous disease, in particular a cancer or a cancerous disease associated with CCR9 expression.

The cancer may be T cell subgroup of acute lymphoblastic leukaemia (T-ALL), prostate, breast, ovarian, non-small cell lung, hepatocellular carcinoma, pancreatic, melanoma, colon, nasopharyngeal carcinoma, Enteropathy-associated T cell lymphoma, hepatosplenic T cell lymphoma, diffuse large B cell lymphoma or mediastinal large B cell lymphoma.

The disease may be a T cell malignancy.

The disease may be leukaemia.

The cancer may be T cell subgroup of acute lymphoblastic leukaemia (T-ALL).

Standard treatments for T-ALL may be chemotherapies, for example one or more of, vincristine, anthracyclines and/or corticosteroids. Clinical remission may be defined as no more than 5 percent of cells in the bone marrow are blast cells, no blast cells present in blood, normal blood cell counts and lack of symptoms.

Suitably, the T-ALL may be relapsed and/or refractory (r/r) T-ALL. Relapsed T-ALL may be defined as patients who have residual leukaemia cells in their bone marrow even after they receive intensive treatment. Refractory T-ALL may be defined as patients who achieve remission but later have decreased numbers of normal blood cells and a return of leukemia cells in their bone marrow. Treatment for relapsed/refractory T-ALL may be more intensive or complex than the treatment used following initial diagnosis.

The disease may be inflammatory bowel disease, Crohn's disease, coeliac disease or graft versus host disease.

Suitably, the CCR9 binding domain or polypeptide of the invention, the CAR of the present invention, the cell of the present invention, the polynucleotide of the invention, the vector of the invention and/or the pharmaceutical composition of the invention may be used for the treatment of a disease associated with CCR9 expression.

Suitably, the CCR9 binding domain or polypeptide of the invention, the CAR of the present invention, the cell of the present invention, the polynucleotide of the invention, the vector of the invention and/or the pharmaceutical composition of the invention may be used for the treatment of T-ALL.

Cells expressing a CAR molecule of the present invention are capable of killing cancer cells, such as T-ALL cells. CAR-expressing cells may either be created ex vivo either from a patient's own peripheral blood (1^{st} party), or in the setting of a haematopoietic stem cell transplant from donor peripheral blood (2^{nd} party), or peripheral blood from an unconnected donor (3^{rd} party). Alternatively, CAR cells may be derived from ex-vivo differentiation of inducible progenitor cells or embryonic progenitor cells to T cells or NK cells. In these instances, CAR cells are generated by introducing DNA or RNA coding for the CAR by one of many means including transduction with a viral vector, transfection with DNA or RNA.

The present invention also relates to a method for the treatment of a disease, such as cancer, which comprises the step of administering the CCR9 binding domain of the invention, the CAR of the present invention, the cell of the present invention, the polynucleotide of the invention, the vector of the invention and/or the pharmaceutical composition of the invention to a subject.

In this respect, the CCR9 binding domain, the CAR, the cell, the polynucleotide, the vector and/or the pharmaceutical composition may be administered to a subject having an existing disease or condition in order to lessen, reduce or improve at least one symptom associated with the disease and/or to slow down, reduce or block the progression of the disease. For example, the method of the invention may cause or promote T cell or NK cell mediated killing of CCR9-expressing cells, such as cancer cells.

The present invention also relates to the use of the CCR9 binding domain of the invention, the CAR of the present invention, the cell of the present invention, the polynucleotide of the invention, the vector of the invention and/or the pharmaceutical composition of the invention in the manufacture of a medicament for treating a disease, such as cancer.

The medical uses and methods of treatment according to the invention encompass the use of the binding domain, polypeptide or CAR of invention in combination with additional active medicaments. For example, the medical uses and methods of treatment of the invention may encompass combinations with known chemotherapeutic, immunomodulatory and/or radiotherapy treatments.

### Second Target Antigens

The medical uses and methods of treatment of the present invention may comprise targeting a second antigen in combination with CCR9. The medical uses and methods of treatment of the present invention may comprise targeting a second and third antigen in combination with CCR9.

The second or third target may be selected from the list comprising: CD1a, CD2, CD3, CD4, CD5, CD7, CD8, CD21, CD30, CD37, CD38, CD70, TRBC1, and TRBC2.

The second target antigen may be CD21.

The second/third antigen may be targeted using the same or a different modality to the modality used to target CCR9. The moieties may be any antigen-binding polypeptide as described herein.

In particular, the anti-CCR9 binding polypeptide may be a CAR according to the present invention. The moiety targeting the second/third target antigens may also be a CAR.

Approaches for targeting multiple antigens using CARs are known in the art; for example a mixture of two pools of single CAR T-cells in which each pool targets a different antigen, two CARs targeting distinct antigens expressed on one T-cell through the use of co-transduction or a bicistronic vector (see e.g. WO2015/075468), tandem CAR T-cells, which contain two distinct binding domains linked to one receptor (see e.g. WO2013/123061).

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### EXAMPLES

### Example 1

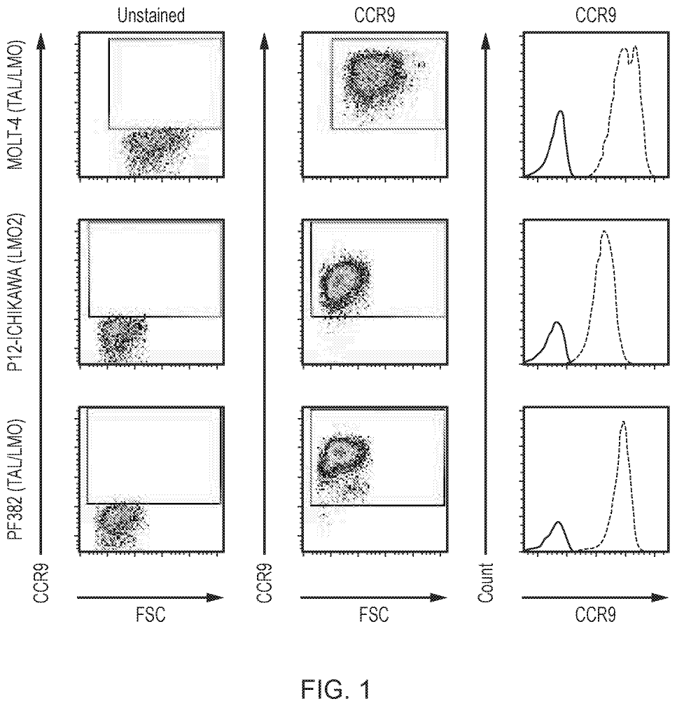

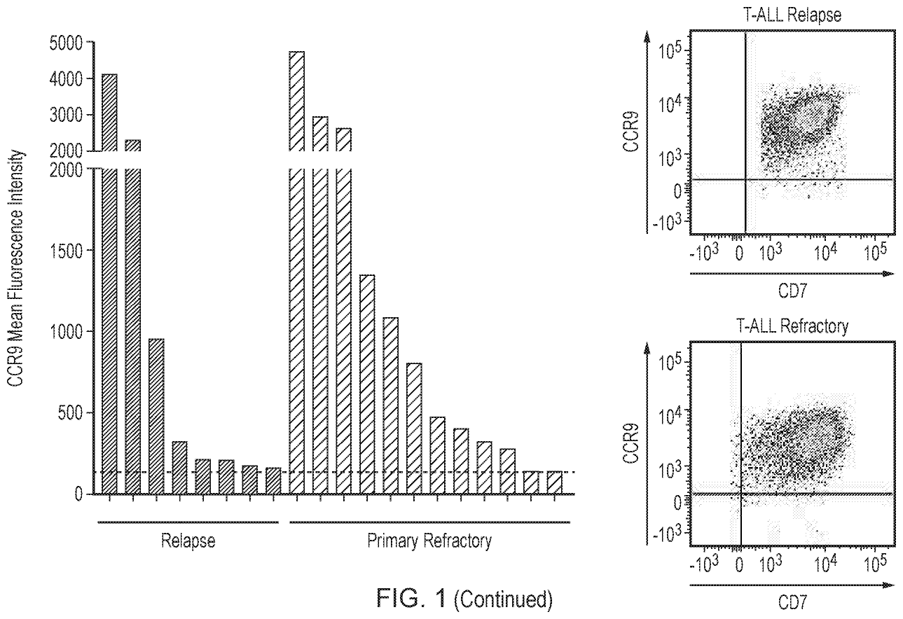

To first determine which antigens are selectively expressed in T-ALL patients but not normal tissue, we analysed the collated gene expression profiles of 35 normal tissues (n=172 samples) as compared to MOLT-4 cells, a TAL1 positive T-ALL cell line included in the Protein Atlas Cancer compendium. Using subtractive transcriptomics, we identified CCR9 as a transcript that was uniquely expressed in MOLT-4 cells but in no other normal tissue. Flow cytometry confirmed high cell surface expression of CCR9 in MOLT-4 cells as well as T-ALL cell lines P12-Ichikawa and PF382 (FIG. 1). Flow cytometry of diagnostic T-ALL samples has confirmed high level cell surface protein expression of CCR9 in the majority of samples examined, and importantly in 4 of 8 relapsed cases and 10 of 12 primary refractory T-ALL samples

### Example 2

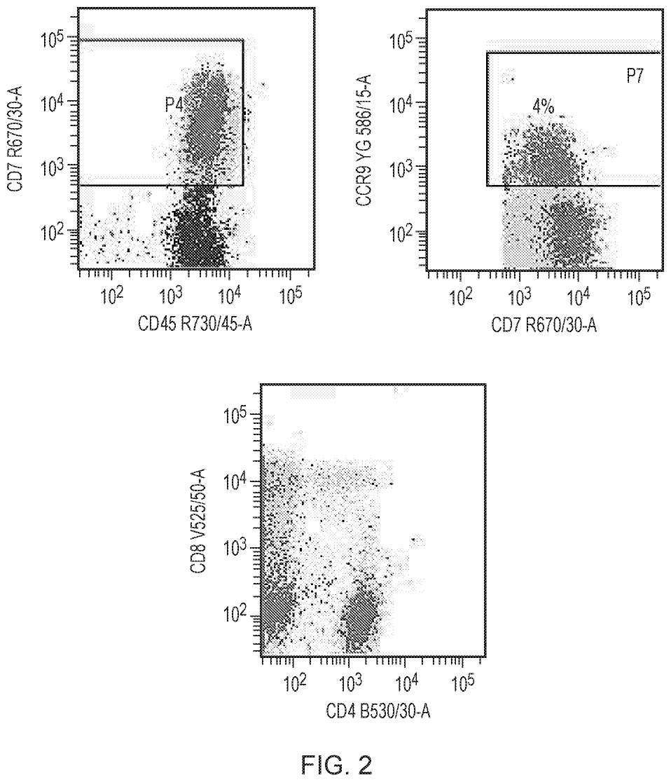

To determine if CCR9 protein was expressed in normal T cells, flow cytometry was performed on peripheral blood from normal donors, and identified a small population of CCR9+ CD4+ cells that constituted <5% of the total T cell population (FIG. 2).

### Example 3

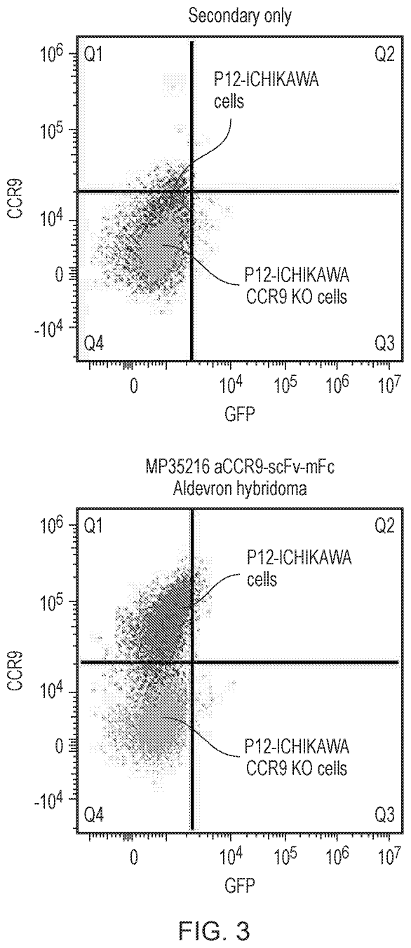

CCR9 Antibody generation: through immunisation of rats, we have generated and characterised a CCR9 antibody (clone 9G7) that binds CCR9 in human T-ALL cells in vitro. When cloned as a single chain variant fragment (scFv), 9G7 binds efficiently to CCR9+ cell line (P12 Ichikawa cells) and is highly specific, with no binding to P12-Ichikawa cells where CCR9 has been previously edited out through CRISPR-Cas9 engineering (FIG. 3).

### Example 4

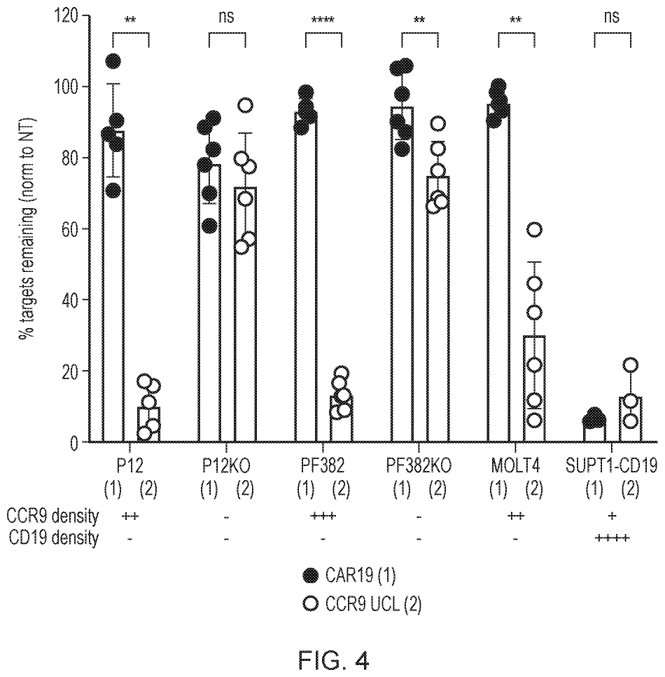

When cloned into CAR T cell format, 9G7 CCR9 directed CAR T cells specifically kill CCR9+ T-ALL cell lines, but not their isogenic CCR9 knockout counterparts (generated through CRISPR CAS9 engineering), showing the target engagement is highly specific (FIG. 4).

### Example 5

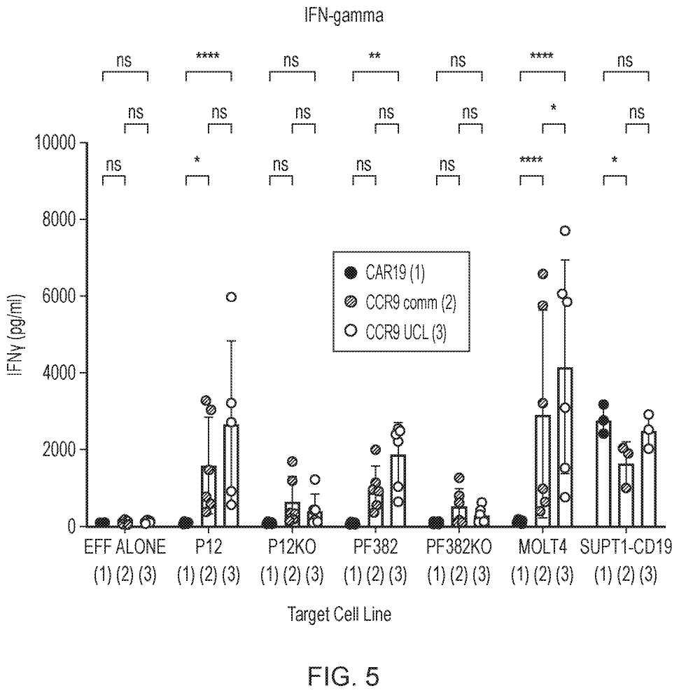

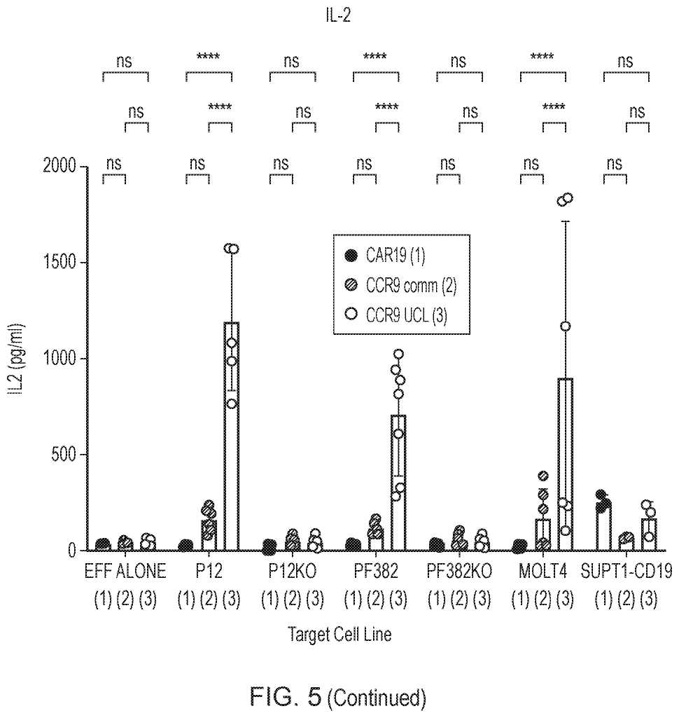

The present CCR9 CAR was compared to a comparator CCR9 binder in CAR format. Flow cytometry and ELISA were used to measure readouts from 48 hour co-cultures with 50 000 targets per well. The present CCR9 CAR resulted in greater cytokine release compared to the existing CCR9 binder (FIG. 5).

### Example 6

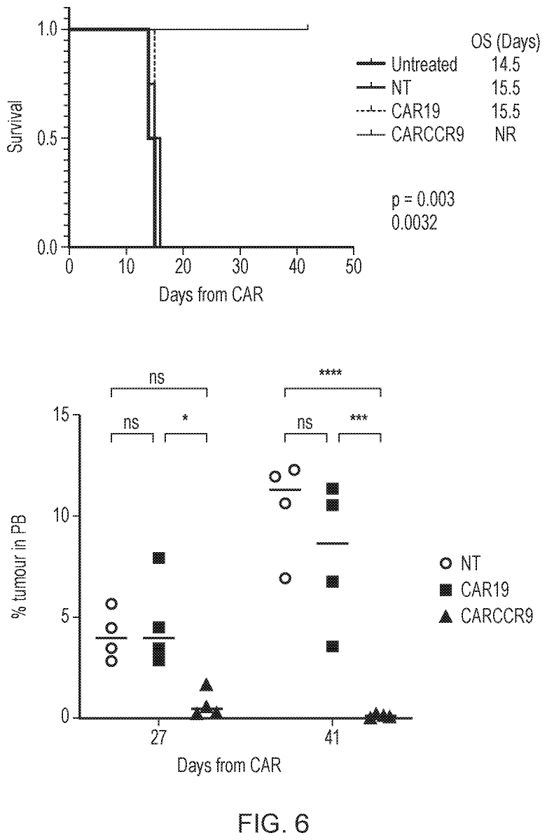

In vivo, 9G7 CCR9 directed CAR T cells lead to sustained tumour responses and prolonged survival in NSG mice engrafted with MOLT4 cells (FIG. 6). Furthermore, 9G7 CCR9 directed CAR T cells completely control dissemination of a primary T-ALL (with low density CCR9 expression) into the peripheral blood.

### Example 7

### Anti-CCR9 CAR has Potent Anti-Leukemic Activity in Patient-Derived Xenograft Models of T-ALL

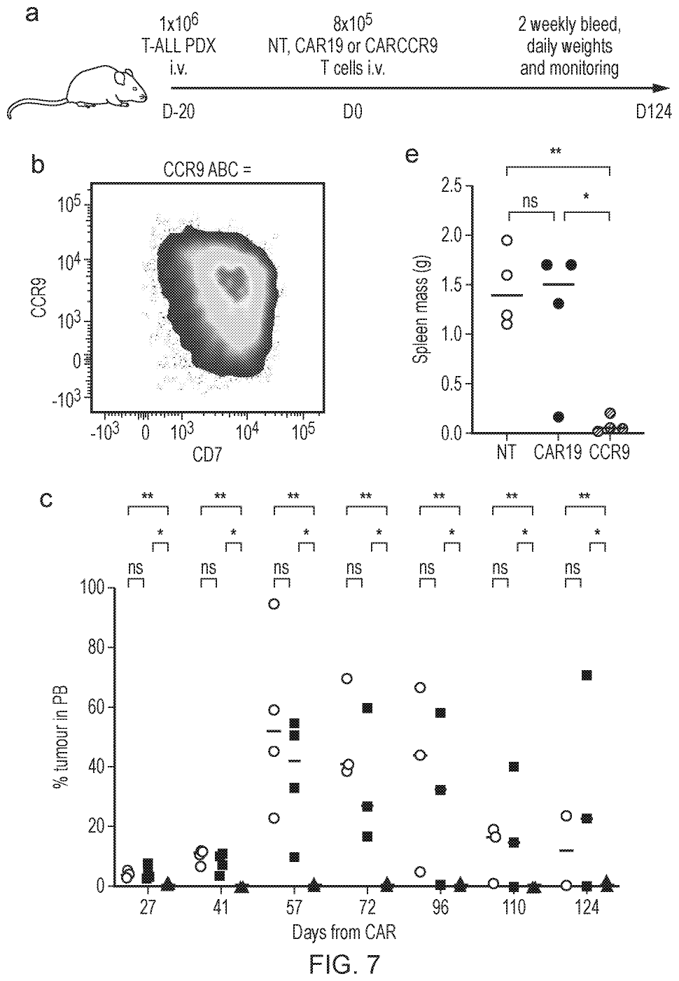

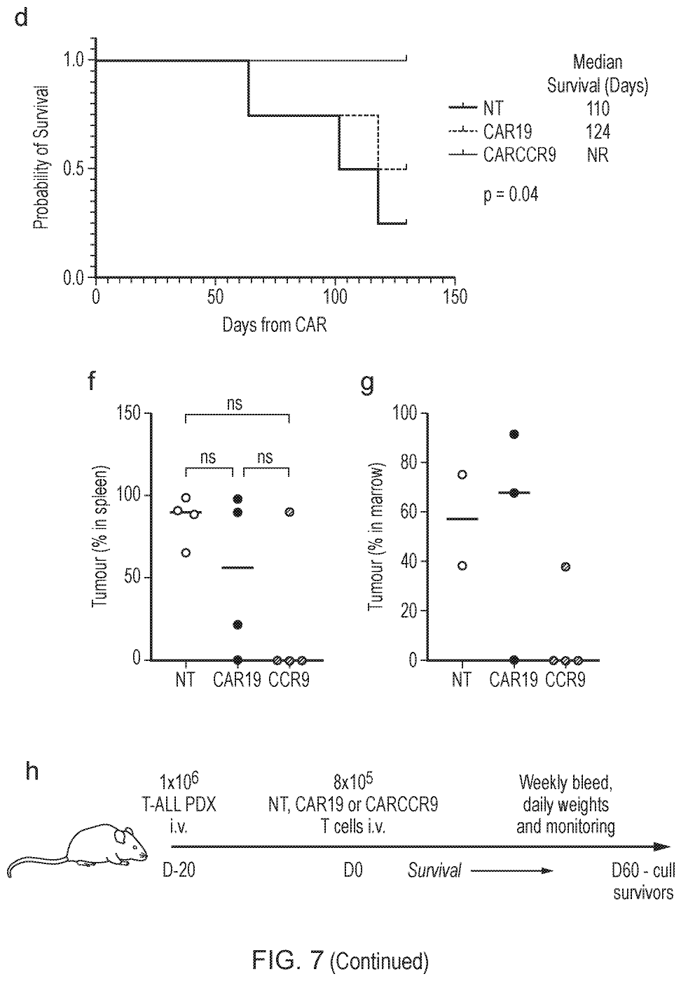

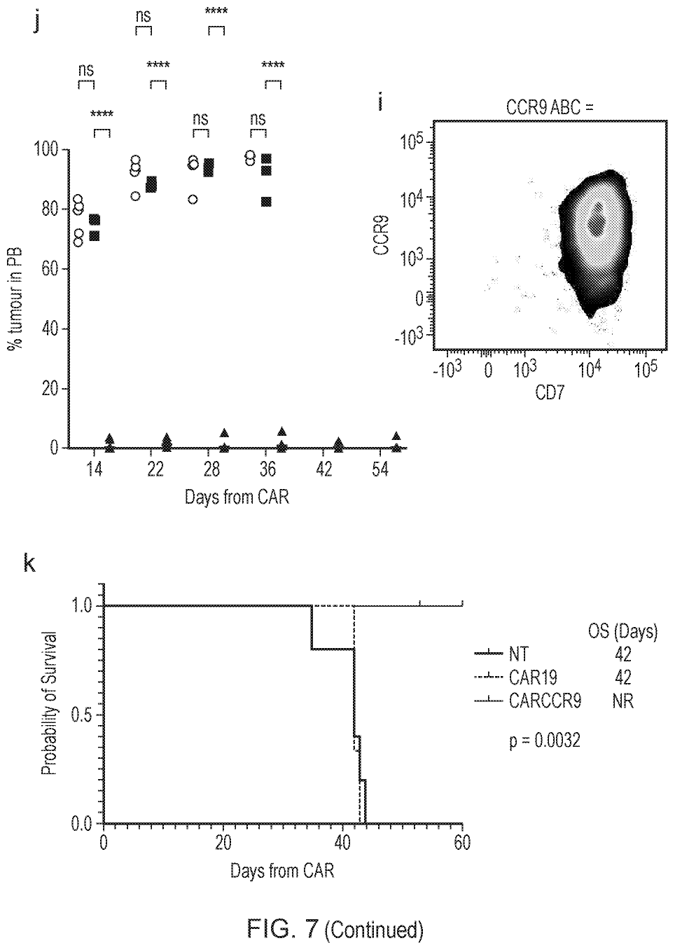

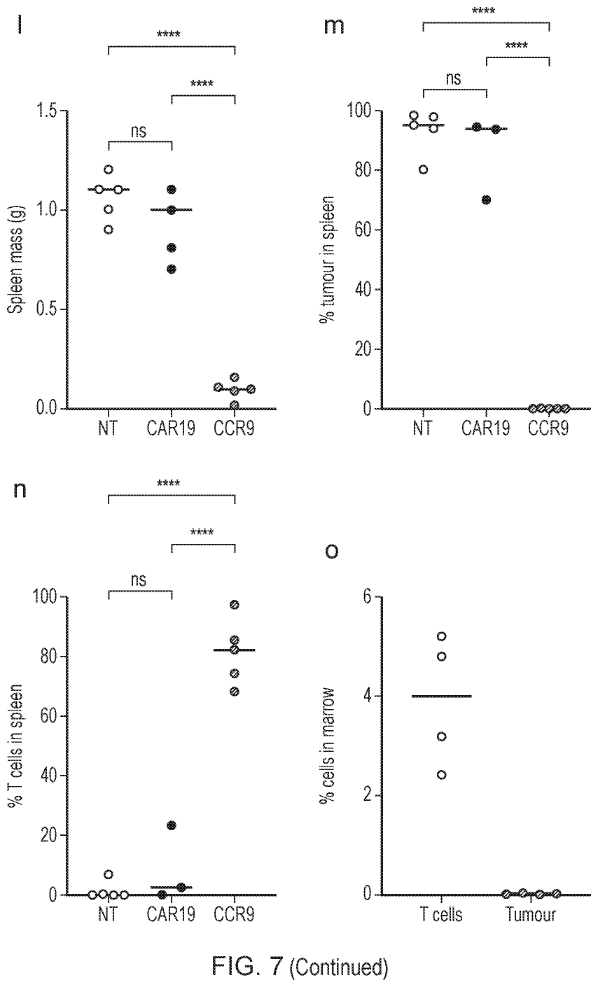

The present CCR9 CAR was further tested in patient-derived xenograft (PDX) models of T-ALL (FIG. 7a-h), with antigen densities of 1078 (FIGS. 7b) and 1139 (FIG. 7i) molecules/cell respectively. NSG mice were injected with 1×10⁶ primary blasts, then 8×10⁵ NT, CAR19 or CCR9 CAR cells were administered IV on D+20 (CAR D+0) (FIG. 7a,h). In PDX model 1078 (FIG. 7a,b), disease was slowly progressive, with NT or CAR19 recipients displayed increasing blast percentage in peripheral blood (FIG. 7c). Leukemic death occurred in most animals by CAR D+120 (FIG. 7d), in association with massive splenomegaly (FIG. 7e) and heavy infiltration of spleen (FIG. 7f)/marrow (FIG. 7g) with T-ALL. Late tumor regression associated with development of xeno-GvHD was seen in one recipient of CAR19. By contrast, in recipients of CCR9 CAR, no tumor was detected in peripheral blood until the end of the study (FIG. 7c), although low level infiltration of T-ALL blasts was seen at necropsy in both spleen and marrow in 1/4 animals (FIG. 7f,g). Blasts were CCR9+ and no T cells were detected. PDX model 1139 was more aggressive (FIG. 7h,i), with recipients of NT or CAR19 displayed increasing ALL burden in peripheral blood over time (FIG. 7j), with eventual leukemic death and massive splenomegaly in all animals (FIG. 7k,l). By contrast, all CCR9 CAR recipients had undetectable leukaemia and disease-free survival until CAR D+60, when mice were culled due to development of graft versus host disease (GvHD) in some animals (median survival NT 42 days, CAR19 42 days, CARCCR9 NR, p=0.0032, FIG. 7k). At the time of cull, all CCR9 CAR recipients had normal-sized spleens, with no detectable leukaemia either in spleen (FIG. 7l, m) or marrow (FIG. 7o). Instead, human T cell infiltration was seen (FIG. 7n,o).

### Example 8

Anti-CCR9 CAR has potent anti-leukemic activity in patient-derived xenograft models of T-ALL with low CCR9 antigen density.

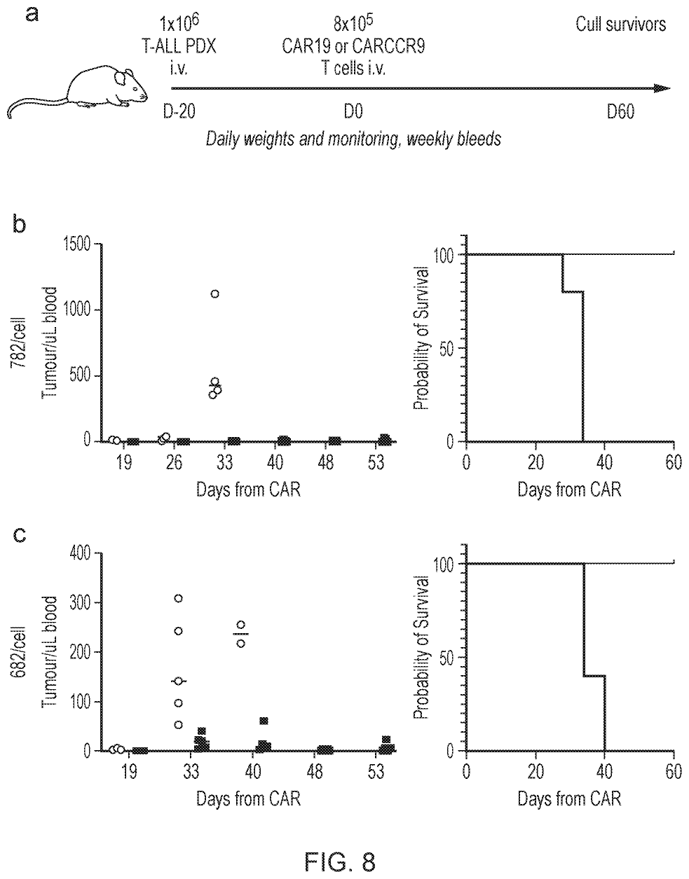

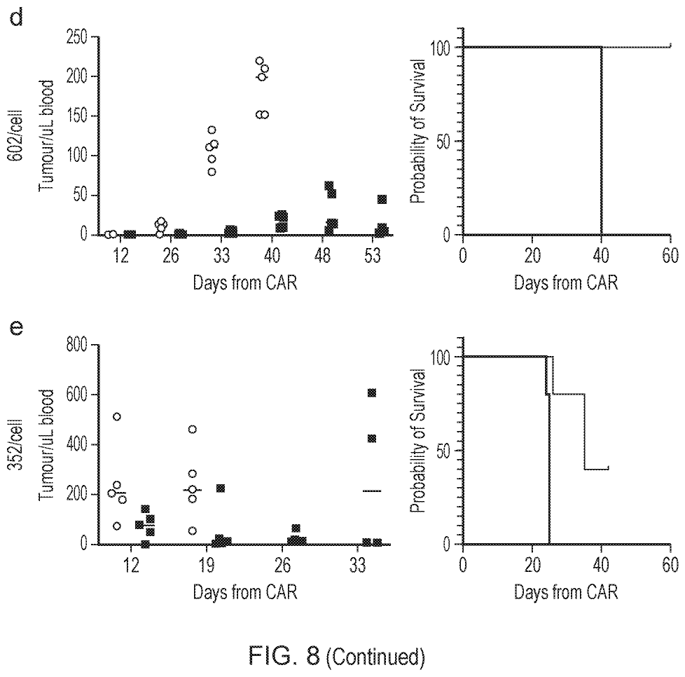

PDX with antigen densities of 782, 682, 602 and 352 CCR9/cell respectively were engrafted in NSG mice as before, with IV administration of 8×105 CAR19 or CCR9 CAR cells on D+20 (FIG. 8a). Even at these low densities, tumour clearance and long term survival until CAR D+60 was seen in all recipients of CCR9 CAR, other than those engrafted with the lowest density PDX (352/cell) (FIG. 8b-e). All surviving mice were culled at D60 due to development of GvHD in the majority of animals. Notably, even in PDX 352, which was the most aggressive model tested, initial disease control was seen in 4/5 recipients of CCR9 CAR, followed by rapid relapse in all mice. This was associated with a survival benefit for CCR9 CAR recipients (FIG. 8e).

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A chimeric antigen receptor (CAR) comprising a CC motif chemokine receptor 9 (CCR9) binding domain.

2. The CAR according to claim 1, wherein the CCR9 binding domain is capable of selectively binding the N-terminal domain of CCR9.

3. The CAR according to claim 1 or 2, wherein the CCR9 binding domain comprises complementarity determining regions (CDRs) comprising the following sequences:
CDR1-
(SEQ ID NO: 1)
GFSVASYD
CDR2-
(SEQ ID NO: 2)
IWANGNT
CDR3-
(SEQ ID NO: 3)
TRGGFAY
CDR4-
(SEQ ID NO: 4)
QSLVHNNGNTY
CDR5-
(SEQ ID NO: 5)
KVS
CDR6-
(SEQ ID NO: 6)
GQGTQYPT;
optionally wherein one or more of the CDRs comprises one, two or three amino acid mutations.

4. The CAR according to any preceding claim, wherein the CCR9 binding domain comprises a variable heavy chain (VH) region comprising the sequence shown as SEQ ID NO: 7 or a variant having at least 80% sequence identity thereto and a variable light chain (VL) region comprising the sequence shown as SEQ ID NO: 8 or a variant of having at least 80% sequence identity thereto.

5. The CAR according to any preceding claim, wherein the CCR9 binding domain comprises the sequence shown as SEQ ID NO: 10 or a variant thereof having at least 80% sequence identity to SEQ ID NO: 10.

6. The CAR according to any preceding claim, which comprises a transmembrane domain selected from the group comprising: a CD8a transmembrane domain; a CD28 transmembrane domain; or a Tyrp-1 transmembrane domain.

7. The CAR according to claim 6, wherein the transmembrane domain comprises one of the sequences selected from the group comprising: SEQ ID NO: 12; SEQ ID NO: 13; or SEQ ID NO: 14; or a variant thereof having at least 80% sequence identity thereto.

8. The CAR according to any of claims 1-7, wherein the CCR9 binding domain and the transmembrane domain are connected by a spacer.

9. The CAR according to claim 8, wherein the spacer comprises one of the following: an lgG1 Fc domain; an IgG1 hinge; an IgG1 hinge-CD8 stalk; a CD8 stalk; a CD2 ectodomain; a CD34 ectodomain; or COMP.

10. The CAR according to claim 8 or claim 9, wherein the spacer comprises one of the sequences selected from the group comprising: SEQ ID NO: 15; SEQ ID NO: 16; SEQ ID NO: 17; SEQ ID NO: 18; SEQ ID NO: 19; SEQ ID NO: 20; SEQ ID NO: 21; or SEQ ID NO: 22; or a variant thereof having at least 80% sequence identity.

11. The CAR according to any of claims 1 to 10 which comprises an intracellular T cell signalling domain which comprises one or more of the following endodomains: CD28 endodomain; 41BB endodomain; OX40 endodomain and CD3-Zeta endodomain.

12. The CAR according to claim 11, wherein the intracellular T cell signalling domain comprises one or more of the sequences selected from the group comprising: SEQ ID NO: 25; SEQ ID NO: 26; SEQ ID NO: 27; SEQ ID NO: 28; SEQ ID NO: 29; SEQ ID NO: 30; or SEQ ID NO: 31; or a variant thereof having at least 80% sequence identity.

13. The CAR according to any one of claims 1 to 12, which comprises the sequence shown as SEQ ID NO: 35 or a variant thereof which has at least 80% sequence identity thereto and retains the capacity to i) bind CCR9 and ii) induce T cell signalling.

14. A polynucleotide comprising a nucleic acid sequence encoding a CAR according to any one of claims 1 to 13.

15. A vector which comprises a polynucleotide according to claim 14.

16. A cell comprising a CAR according to any of claims 1 to 13.

17. A cell comprising a polynucleotide according to claim 14, or a vector according to claim 15.

18. The cell according to claim 16 or 17, wherein the cell is a T cell or an NK cell.

19. A method for making a cell according to any of claims 16 to 18, which comprises the step of introducing a polynucleotide according to claim 14 or a vector according to claim 15 into said cell.

20. A pharmaceutical composition which comprises a CAR according to any of claims 1 to 13, a polynucleotide according to claim 14, a vector according to claim 15, or a cell according to any one of claims 16 to 18, together with a pharmaceutically acceptable carrier, diluent or excipient.

21. A CAR according to any one of claims 1 to 13, a polynucleotide according to claim 14, a vector according to claim 15, a cell according to any of claims 16 to 18, or a pharmaceutical composition according to claim 20 for use as a medicament in the treatment of a disease.

22. A method for treating a disease, which comprises the step of administering a CAR according to any one of claims 1 to 13, a polynucleotide according to claim 14, a vector according to claim 15, a cell according to any of claims 16 to 18, or a pharmaceutical composition according to claim 20 to a subject.

23. Use of a CAR according to any one of claims 1 to 13, a polynucleotide according to claim 14, a vector according to claim 15, a cell according to any of claims 16 to 18, or a pharmaceutical composition according to claim 20 in the manufacture of a medicament for treating a disease.

24. The CAR, the polynucleotide, the vector, the cell, or the pharmaceutical composition for the use according to claim 21; or the method according to claim 22; or the use according to claim 23; wherein the disease is cancer.

25. The CAR, the polynucleotide, the vector, the cell, or the pharmaceutical composition for the use, or the method, or the use according to claim 24, wherein the cancer is T-ALL.


SEQUENCE LISTING (35 sequences)

SEQ ID NO: 1 - protein, 8 aa, synthetic construct
GFSVASYD

SEQ ID NO: 2 - protein, 7 aa, synthetic construct
IWANGNT

SEQ ID NO: 3 - protein, 7 aa, synthetic construct
TRGGFAY

SEQ ID NO: 4 - protein, 11 aa, synthetic construct
QSLVHNNGNTY

SEQ ID NO: 5 - skipped

SEQ ID NO: 6 - protein, 8 aa, synthetic construct
GQGTQYPT

SEQ ID NO: 7 - protein, 113 aa, synthetic construct
QVQLKESGPGLVQPSQTLSLTCTVSGFSVASYDMHWVRLPPGKGLEWMGIIWANGNTHYNSGLKSRLSISRDTSKSQVFLKMNSLQTEDTAIYFCTRGGFAYWGQGTLVTVSS

SEQ ID NO: 8 - protein, 111 aa, synthetic construct
DVVMTQTPVSLSVSLGGQVSISCRSSQSLVHNNGNTYLSWYLQKPGQSPQLLIYKVSNRFSGVSDRFSGSGSGTDFTLKISRVEPDDLGVYYCGQGTQYPTFGGGTKLELK

SEQ ID NO: 9 - protein, 16 aa, synthetic construct
SGGGGSGGGGSGGGGS

SEQ ID NO: 10 - protein, 240 aa, synthetic construct
QVQLKESGPGLVQPSQTLSLTCTVSGFSVASYDMHWVRLPPGKGLEWMGIIWANGNTHYNSGLKSRLSISRDTSKSQVFLKMNSLQTEDTAIYFCTRGGFAYWGQGTLVTVSASGGGGSGGGGSGGGGSDVVMTQTPVSLSVSLGGQVSISCRSSQSLVHNNGNTYLSWYLQKPGQSPQLLIYKVSNRFSGVSDRFSGSGSGTDFTLKISRVEPDDLGVYYCGQGTQYPTFGGGTKLELK

SEQ ID NO: 11 - protein, 369 aa, synthetic construct
MTPTDFTSPIPNMADDYGSESTSSMEDYVNFNFTDFYCEKNNVRQFASHFLPPLYWLVFIVGALGNSLVILVYWYCTRVKTMTDMFLLNLAIADLLFLVTLPFWAIAAADQWKFQTFMCKVVNSMYKMNFYSCVLLIMCISVDRYIAIAQAMRAHTWREKRLLYSKMVCFTIWVLAAALCIPEILYSQIKEESGIAICTMVYPSDESTKLKSAVLTLKVILGFFLPFVVMACCYTIIIHTLIQAKKSSKHKALKVTITVLTVFVLSQFPYNCILLVQTIDAYAMFISNCAVSTNIDICFQVTQTIAFFHSCLNPVLYVFVGERFRRDLVKTLKNLGCISQAQWVSFTRREGSLKLSSMLLETTSGALSL

SEQ ID NO: 12 - protein, 21 aa, synthetic construct
IYIWAPLAGTCGVLLLSLVIT

SEQ ID NO: 13 - protein, 27 aa, synthetic construct
FWVLVVVGGVLACYSLLVTVAFIIFWV

SEQ ID NO: 14 - protein, 24 aa, synthetic construct
IIAIAVVGALLLVALIFGTASYLI

SEQ ID NO: 15 - protein, 234 aa, synthetic construct
AEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKD

SEQ ID NO: 16 - protein, 45 aa, synthetic construct
TTTPAPRPPTPAPTIASQPLSLRPEACRPAAGGAVHTRGLDFACD

SEQ ID NO: 17 - protein, 20 aa, synthetic construct
AEPKSPDKTHTCPPCPKDPK

SEQ ID NO: 18 - protein, 237 aa, synthetic construct
AEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 19 - protein, 236 aa, synthetic construct
AEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 20 - protein, 185 aa, synthetic construct
KEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLD

SEQ ID NO: 21 - protein, 259 aa, synthetic construct
SLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKT

SEQ ID NO: 22 - protein, 45 aa, synthetic construct
DLGPQMLRELQETNAALQDVRELLRQQVREITFLKNTVMECDACG

SEQ ID NO: 23 - protein, 5 aa, synthetic construct
CDACG

SEQ ID NO: 24 - protein, 20 aa, synthetic construct
QQVREITFLKNTVMECDACG

SEQ ID NO: 25 - protein, 39 aa, synthetic construct
RSKRSRLLHSDYMNMTPRRPGPTRKHYQPYAPPRDFAAY

SEQ ID NO: 26 - protein, 37 aa, synthetic construct
RRDQRLPPDAHKPPGGGSFRTPIQEEQADAHSTLAKI

SEQ ID NO: 27 - protein, 42 aa, synthetic construct
KRGRKKLLYIFKQPFMRPVQTTQEEDGCSCRFPEEEEGGCEL

SEQ ID NO: 28 - protein, 114 aa, synthetic construct
RSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 153 aa, synthetic construct
RSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 189 aa, synthetic construct
RSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 155 aa, synthetic construct
KRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRA

SEQ ID NO: 32 - protein, 20 aa, synthetic construct
METDTLLLWVLLLWVPGSTG

SEQ ID NO: 33 - protein, 402 aa, synthetic construct
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSAS

SEQ ID NO: 34 - protein, 157 aa, synthetic construct
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 35 - protein, 487 aa, synthetic construct
METDTLLLWVLLVWIPGSTGQVQLKESGPGLVQPSQTLSLTCTVSGFSVASYDMHWVRLPPGKGLEWMGIIWANGNTHYNSGLKSRLSISRDTSKSQVFLKMNSLQTEDTAIYFCTRGGFAYWGQGTLVTVSSSGGGGSGGGGSGGGGSDVVMTQTPVSLSVSLGGQVSISCRSSQSLVHNNGNTYLSWYLQKPGQSPQLLIYKVSNRFSGVSDRFSGSGSGTDFTLKISRVEPDDLGVYYCGQGTQYPTFGGGTKLELKRSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR